SEQUENCE LISTING (34 sequences)

SEQ ID NO: 1 - DNA, 20 bp, Artificial Sequence
  IL-8(NM_000584.3) Forward primer
ggccgtggctctcttggcag

SEQ ID NO: 2 - DNA, 21 bp, Artificial Sequence
  IL-8(NM_000584.3) Reverse primer
tgtgttggcgcagtgtggtcc

SEQ ID NO: 3 - DNA, 21 bp, Artificial Sequence
  CXCR1(NM_000634.2) Forward primer
gagcccccgaatctgacatta

SEQ ID NO: 4 - DNA, 21 bp, Artificial Sequence
  CXCR1(NM_000634.2) Reverse primer
agtgcctgcctcaatgtctcc

SEQ ID NO: 5 - DNA, 20 bp, Artificial Sequence
  VEGF(NM_001025366.2) Forward primer
gggcagaatcatcacgaagt

SEQ ID NO: 6 - DNA, 20 bp, Artificial Sequence
  VEGF(NM_001025366.2) Reverse primer
tggtgatgttggactcctca

SEQ ID NO: 7 - DNA, 20 bp, Artificial Sequence
  KDR(NM_002253.2) Forward primer
atgctggactgctggcacgg

SEQ ID NO: 8 - DNA, 20 bp, Artificial Sequence
  KDR(NM_002253.2) Reverse primer
tcacaggccggctctttcgc

SEQ ID NO: 9 - DNA, 20 bp, Artificial Sequence
  HGF(NM_00601.4) Forward primer
ctggttccccttcaatagca

SEQ ID NO: 10 - DNA, 20 bp, Artificial Sequence
  HGF(NM_00601.4) Reverse primer
ctccagggctgacatttgat

SEQ ID NO: 11 - DNA, 20 bp, Artificial Sequence
  c-Met(NM_001127500.1) Forward primer
ccaatggcctgcagccgtga

SEQ ID NO: 12 - DNA, 20 bp, Artificial Sequence
  c-Met(NM_001127500.1) Reverse primer
ctgttctggggctgccgctc

SEQ ID NO: 13 - DNA, 20 bp, Artificial Sequence
  MMP-9(NM_004994.2) Forward primer
caacatcacctattggatcc

SEQ ID NO: 14 - DNA, 20 bp, Artificial Sequence
  MMP-9(NM_004994.2) Reverse primer
cgggtgtagagtctctcgct

SEQ ID NO: 15 - DNA, 20 bp, Artificial Sequence
  GAPDH(NM_002046.3) Forward primer
gagtcaacggatttggtcgt

SEQ ID NO: 16 - DNA, 20 bp, Artificial Sequence
  GAPDH(NM_002046.3) Reverse primer
gacaagcttcccgttctcag

SEQ ID NO: 17 - DNA, 20 bp, Artificial Sequence
  Insulin(NM_000207) Forward primer
cctgtgcggctcacacctgg

SEQ ID NO: 18 - DNA, 20 bp, Artificial Sequence
  Insulin(NM_000207) Reverse primer
ccactcaggcaggcagccac

SEQ ID NO: 19 - DNA, 23 bp, Artificial Sequence
  Glut2(L09674) Forward primer
aggacttctgtggaccttatgtg

SEQ ID NO: 20 - DNA, 20 bp, Artificial Sequence
  Glut2(L09674) Reverse primer
gttcatgtcaaaaagcaggg

SEQ ID NO: 21 - DNA, 25 bp, Artificial Sequence
  Pdx1(NM_000209) Forward primer
ggatgaagtctaccaaagctcacgc

SEQ ID NO: 22 - DNA, 25 bp, Artificial Sequence
  Pdx1(NM_000209) Reverse primer
ccagatcttgatgtgtctctcggtc

SEQ ID NO: 23 - DNA, 23 bp, Artificial Sequence
  Ngn3(AF234829) Forward primer
cgtgaactccttgaactgagcag

SEQ ID NO: 24 - DNA, 22 bp, Artificial Sequence
  Ngn3(AF234829) Reverse primer
tggcactcctgggacagatttc

SEQ ID NO: 25 - DNA, 20 bp, Artificial Sequence
  Nkx6.1(NM_006168.2) Forward primer
caatggagggcacccggcag

SEQ ID NO: 26 - DNA, 20 bp, Artificial Sequence
  Nkx6.1(NM_006168.2) Reverse primer
ccagaagatgggcgtccggc

SEQ ID NO: 27 - DNA, 24 bp, Artificial Sequence
  PC2(NM_002594) Forward primer
gcatcaagcacagacctacactcg

SEQ ID NO: 28 - DNA, 22 bp, Artificial Sequence
  PC2(NM_002594) Reverse primer
gagacacaacccttcatccttc

SEQ ID NO: 29 - DNA, 27 bp, Artificial Sequence
  PC1/3(NM_000439) Forward primer
ttggctgaaagagaacgggatacatct

SEQ ID NO: 30 - DNA, 27 bp, Artificial Sequence
  PC1/3(NM_000439) Reverse primer
acttctttggtgattgctttggcggtg

SEQ ID NO: 31 - DNA, 21 bp, Artificial Sequence
  SUR1(NM_000352.3) Forward primer
cacatccaccacagcacatgg

SEQ ID NO: 32 - DNA, 25 bp, Artificial Sequence
  SUR1(NM_000352.3) Reverse primer
gtcttgaagaagatgtatctcctca

SEQ ID NO: 33 - DNA, 20 bp, Artificial Sequence
  GAPDH(NM_002046) Forward primer
caaattcgttgtcataccag

SEQ ID NO: 34 - DNA, 18 bp, Artificial Sequence
  GAPDH(NM_002046) Reverse primer
cgtggaaggactcatgac

(19) United States
(12) Patent Application Publication
(10) Pub. No.: US 2015/0157663 A1
(43) Pub. Date: Jun. 11, 2015

(54) Pharmaceutical Composition Comprising Human-Blood-Derived-Cell Mass

(71) Applicant: SNU R&DB FOUNDATION, Seoul (KR)

(72) Inventors: Young Bae Park, Seoul (KR); Hyo Soo Kim, Seoul (KR); Jin Hur, Seoul (KR); Jae Il Choi, Seoul (KR); Ji Yeon Yun, Gyeonggi-do (KR)

(21) Appl. No.: 14/372,891

(22) PCT Filed: Jan. 18, 2013
(86) PCT No.: PCT/KR2013/000441

§ 371 Date: Jul. 17, 2014

(30) Foreign Application Priority Data
Jan. 19, 2012 (KR) 10-2012-0006070
Aug. 8, 2012 (KR) 10-2012-0086956
Aug. 8, 2012 (KR) 10-2012-0086957
Aug. 8, 2012 (KR) 10-2012-0086958
Aug. 8, 2012 (KR) 10-2012-0086959
Jan. 18, 2013 (KR) 10-2013-0005902

(51) Int. Cl. A61K 35/15; A61K 35/19; A61K 35/12

(52) U.S. Cl. CPC A61K 35/15; A61K 35/12; A61K 35/19

(57) ABSTRACT

There are provided a pharmaceutical composition for treating immune-related diseases, a pharmaceutical composition for treating ischemic diseases, a pharmaceutical composition for promoting lymphangiogenesis, a pharmaceutical composition for treating neurological diseases, a pharmaceutical composition for treating metabolic diseases, and the like, which contain blood-born hematospheres, and more specifically, may differentiate into inflammatory mononuclear cells, vascular endothelial cells, vascular smooth muscle cells, lymphatic vessel adult stem cells and progenitor cells, neural progenitor cells and nerve cells, insulin secreting cells, and the like by effective 3D culturing using blood mononuclear cells. Therefore, the present invention is expected to be used for development of a cell therapeutic agent for various types of diseases. Also, when blood-born hematospheres according to the present invention are used, it is possible to address previous problems associated with development of a stem cell therapeutic agent such as tumor occurrence, immune rejection, ethical issues, and difficult differentiation methods.

### TECHNICAL FIELD

The present invention relates to a pharmaceutical composition for cell treatment using blood-born hematospheres and a method of treating diseases including immune-related diseases, ischemic diseases, neurological diseases, and metabolic diseases using the composition.

### BACKGROUND ART

Stem cells refer to fundamental cells of cells or tissues forming a subject and cells that are repeatedly divided and have a self-renewal capacity and a multilineage differentiation potential to differentiate into cells having a specific function depending on environments. Also, according to types of differentiable cells, stem cells include totipotent stem cells generated when a fertilized egg begins a first division, pluripotent stem cells in an inner membrane of a blastocyst generated by repeated divisions of the cells, and multipotent stem cells in mature tissues and organs.

A representative example of the totipotent stem cells is a fertilized egg having a sufficient ability to grow into a subject when it is implanted in a uterus. The totipotent stem cells refer to cells having totipotency that can generate all structures (fetus and placenta) necessary for fetal growth. The pluripotent stem cells are cells at a slightly more advanced stage in development than the fertilized egg and include an inner cell mass (ICM) of a developing blastocyst. The ICM is a cell population which may form a body of the fetus later and is distinguished from outer trophoblast which may form a placenta. When only the ICM is put into the uterus, no placenta is formed and thereby no fetus is developed. However, the ICM still has an ability to differentiate into all types of cells forming a body of the fetus. Embryonic stem cells are obtained by culturing the ICM while maintaining pluripotency.

The multipotent stem cells are visible after development has advanced further, and have cell fates to differentiate into specific systems that are set to some extent. These cells are present in children and adults in addition to the fetus, continuously replace cells in tissues having a rapid cell replacement cycle, and include, for example, hematopoietic stem cells in bone marrow and undifferentiated cells of epithelial tissues forming a digestive system wall. In the central nervous system of adults, which is known to have no regenerative ability, the presence of multipotent stem cells was identified. Since the multipotent stem cells may be obtained from adults, there are no ethical issues. Since types of differentiated cells are already limited compared to embryonic stem cells, it is easy to obtain cells having a specific phenotype.

Research on development of homogeneous human cells and tissues using characteristics of embryo and pluripotent stem cells is currently underway by global life sciences companies. The target multipotent stem cells mainly studied in companies include hematopoietic stem cells, hematopoietic stem cells, neural stem cells, mesenchymal stem cells, and the like. The hematopoietic stem cells differentiate into lymphocytes, white blood cells, red blood cells, and the like after bone marrow transplantation. The neural stem cells become cells forming nervous tissue such as neurons, stellate cells, oligodendrocytes, and the like.

The immortality and multi differentiation of the stem cells provide a good in vitro model for studying the development process of humans. Also, development of a new drug may be initiated when a drug test and a toxicity test are performed on homogeneous human tissues or cells obtained from the stem cells. A large amount of cells or tissues that can replace damaged tissues may be obtained, and thereby the stem cells are expected to be used for treating intractable diseases.

Among the stem cells, bone marrow-derived stem cells are considered as an ultimate treatment method of diseases such as blood cancer, lymphoma, and bone marrow suppression. However, there is a problem in that bone marrow needs to be directly collected. Recently, a method in which stem cells in bone marrow migrate into the blood by G-CSF injection has been used, but this method has potential side effects of the G-CSF drug itself.

Meanwhile, stem cells included in cord blood are actively frozen and stored, and stored in cord blood banks. However, this is not widely performed. In order to secure a sufficient number of adult stem cells in blood under the above circumstance, development of technology for in vitro adult stem cells in blood is an important subject in the related art.

In most techniques for amplifying in vitro adult stem cells in blood in the related art, artifacts were used to artificially create an environment in bone marrow in many cases, and a partial success was obtained (Peerani R, Zandstra P W. Enabling stem cell therapies through synthetic stem cell-niche engineering. The Journal of Clinical Investigation. 2010; 120: 60-70). In the environment of bone marrow of adult stem cells in blood, since contacts with various supporting cells, cytokines provided therefrom, and an extracellular matrix are included, it is difficult to provide an environment in which proliferation of adult stem cells in blood is sufficiently promoted using such artifacts.

Meanwhile, in research on adult tissue-derived stem cells in the related art, several researchers reported a successful method in which brain tissue or cardiac tissue-derived cells are cultured in a spherical shape and thereby the number of stem cells is amplified or pluripotency is maintained (Caldwell M A, He X, Wilkie N, et al. Growth factors regulate the survival and fate of cells derived from human neurospheres. Nat Biotech. 2001; 19:475-479 and Messina E, De Angelis L, Frati G, et al. Isolation and expansion of adult cardiac stem cells from human and murine heart. Circ Res. 2004; 95:911-921).

As described above, bone marrow-derived stem cells may differentiate into several cells forming body organs and may also differentiate into immune cells that can protect host cells from pathogens or viruses which may infect in vitro. Among these, anti-inflammatory monocytes, type 2 T lymphocytes (Th2), and regulatory lymphocytes (Treg) are very important for a body immune system, and are particularly important lymphocytes, indispensable for an adaptive immune system. It is known that the cells do not directly attack and kill host cells infected with viruses and pathogens, but help other immune cells to exhibit functions thereof. Also, these cells are very important in producing antibodies, and amplification and activation cytotoxic T cells by B lymphocytes (B cells), and are very important cells that are essential for phagocytes and macrophases to perform phagocytosis. The importance thereof may also be determined in human immunodeficiency viruses. The specific virus infects and destroys type 2 CD4 T lymphocytes, and the infected host finally has acquired immune deficiency syndrome. Also, regulatory lymphocytes as well as other lymphocytes suppress a body immune system, suppress an immune response that may be induced by autoantigens, and allow immune tolerance against the body's immune responses. In addition, since these help autoimmune diseases to be down-regulated, a great deal of research on roles thereof is currently underway in the study of autoimmune diseases and cancer.

On the other hand, since study of type 2 T lymphocytes and regulatory lymphocytes in humans is very difficult, researchers study functions thereof mainly using rodents.

Also, research on treatment of ischemic diseases by differentiating the stem cells into vascular progenitor cells, vascular endothelial cells, and vascular smooth muscle cells is underway.

Meanwhile, lymphatic vessels form complex mesh tissues, migrate an interstitial fluid to between tissues and blood vessels, and are important for maintaining homeostasis, metabolism, and immune functions. Also, the lymphatic vessels are involved in various types of pathophysiology such as cancer, lymphedema, and inflammatory reactions. Therefore, there is a growing interest in technology for securing lymphatic vessel adult stem cells in blood and progenitor cells.

Lymphangiogenesis occurring in adults spread existing lymphatic vessels and neighboring monocyte cells help or are directly involved in the process of lymphangiogenesis (The crucial role of macrophages in lymphangiogenesis. J. Clin. Invest. 2005:115:2316-2319 2005). As research on lymphangiogenesis in adults in the related art, a method in which cells having a lymphatic vessel marker among bone marrow cells of a lower animal (mouse) are cultured in vitro to amplify the number of stem cells or maintain pluripotency has been reported (Podoplanin-Expressing Cells Derived From Bone Marrow Play a Crucial Role in Postnatal Lymphatic Neovascularization. Circulation. 2010; 122; 1413-1425).

Meanwhile, research on bone marrow-derived stem cells in humans is regarded as an ultimate treatment method of diseases such as lymphoma, but there is a problem in that the stem cells need to be directly collected. Recently, a method in which stem cells in bone marrow move into blood by G-CSF injection has been used, but this method has potential side effects of the G-CSF drug itself. Also, cord blood-derived stem cells are actively frozen and stored, and stored in cord blood banks. However, this is not widely performed.

In order to secure a sufficient number of lymphatic vessel adult stem cells in blood and progenitor cells under the above circumstance, development of technology for amplifying lymphatic vessel adult stem cells in blood and progenitor cells in vitro is an important subject in the related art.

In addition, there is an attempt to use stem cells for neurological disease treatment. Neurodegenerative diseases are caused by permanent destruction or dysfunction of nerve cells of a central nervous system. Neurodegenerative diseases cause serious social problems. When brain nervous tissues are damaged, a recovering ability thereof is very limited. Therefore, a fundamental treatment method of neurodegenerative diseases is not yet developed. That is, up to now, according to a theory that regeneration of a central nervous system is impossible, drugs, enzymes, and the like are administered systematically in order to treat intractable neurological diseases by supplementing deficient neurotransmitters. Also, due to the blood-brain-barrier, movement to a desired location is difficult. Recently, various viral vectors have been used for gene therapy in nerve cells, but this method is difficult to apply to extensive lesions and has limitations in reconstruction of already damaged nerve cells and circuits.

As a new method of treating such intractable brain diseases, research on stem cell treatment is being actively performed. Stem cell treatment proposes a possibility in that damaged and disappeared cells are replaced, necessary neurotransmitters are secreted, and a neural circuit may be eventually regenerated. Therefore, there is a growing interest in its therapeutic usefulness.

In addition, research on differentiation of the stem cells into insulin is being actively performed (A. S. Boyd, K. J. Wood, Characteristics of the early immune response following transplantation of mouse ES cell derived insulin-producing cell clusters, PLoS One, 2010).

Despite the above advantages, there are several problems when stem cells are actually applied to treatment. For example, embryo stem cells (ESCs) actively proliferate and may differentiate into all types of cells, but there are unsolved problems such as tumor occurrence due to continuous proliferation, immune rejection response problems, and ethical issues. In order to solve problems of the embryo stem cells (ESCs), induced pluripotent stem cells (IPSs) were developed in 2006 (Kazutoshi Takahashi and Shinya Yamanaka, Induction of Pluripotent Stem Cells from Mouse Embryonic and Adult Fibroblast Cultures by Defined Factors. Cell, 2006; 126:663-676). However, there are still several problems when IPSs are actually used for treatment, particularly, for example, use of viruses and immune rejection responses. Also, adult stem cells (ASCs) have problems in that the number of cells (ASCs) is small, proliferation is not active, and a method of differentiating into each cell is difficult.

### DISCLOSURE

### Technical Problem

In view of the above problems in the related art, the inventors have attempted to develop a culturing method in which anti-inflammatory monocytes, type 2 T lymphocytes, and regulatory lymphocytes, which are collected and amplified in a specific portion of a body under specific conditions, are amplified in vitro using human blood-derived mononcyte cells by a high density 3D culturing method, and functions thereof may be finally studied on humans rather than rodents, and have completed the present invention.

Anti-inflammatory monocytes, type 2 T lymphocytes, and regulatory T lymphocytes are obtained by killing rodents for cell treatment or research, and are not applied to humans as a final target. However, the present invention addressed these problems. The present invention provides a new preparation method in which anti-inflammatory monocytes, type 2 T lymphocytes, and regulatory T lymphocytes are cultured and proliferated in vitro, and researchers may very conveniently easily study functions thereof on human subjects. Eventually, the present invention provides a method of treating immune-related diseases using blood-born hematospheres.

Also, the inventors have attempted to address limitations such as tumor occurrence, immune rejection, and ethical issues which are problems of stem cell therapeutic agents such as previously developed embryo stem cells (ESCs), induced pluripotent stem cells (IPSs), and adult stem cells (ASCs), found a possibility of angiogenesis through vascular progenitor cell amplification and vascular endothelial cell and vascular smooth muscle cell differentiation using blood-born hematospheres, and have completed the present invention. The present invention provides a method of treating ischemic diseases using blood derived hematospheres.

In addition, the inventors have amplified lymphatic vessel adult stem cells in blood and progenitor cells in vitro, which are present in blood in small amounts, using human blood-derived monocyte cells by a high density 3D culturing method and attempted to develop an optimal autologous treatment method of cell treatment of lymphatic vessel-related diseases.

Therefore, the present invention provides a method in which a large amount of lymphatic vessel adult stem cells and progenitor cells is cultured and proliferated by in vitro culturing of autologous monocytes in blood, and eventually provides a method of treating lymphatic diseases such as lymphatic dysplasia using the same.

Also, the inventors have isolated monocytes from human blood, generated blood-born hematospheres, and created an environment similar to an actual body. When the outcome is applied to a specific culturing condition, a possibility of differentiation into nervous system cells was found, and the inventors have completed the present invention.

Therefore, the present invention provides a method of treating neurological diseases using blood-born hematospheres.

In addition, in order to address problems of a previously developed stem cell therapeutic agent using embryo stem cells (ESCs), induced pluripotent stem cells (IPSs), adult stem cells (ASCs), and the like, such as tumor occurrence, immune rejection, ethical issues, and differentiation methods, the present invention provides a method in which blood-born hematospheres (BBHSs) are induced to differentiate into insulin secreting cells and used as a therapeutic agent for metabolic diseases.

The scope of the present invention is not limited to the above-described objects, and other unmentioned objects may be clearly understood by those skilled in the art from the following descriptions

### Technical Solution

The present invention provides a pharmaceutical composition for treating immune-related diseases, containing blood-born hematospheres generated when monocyte cells are isolated from human blood and then 3D aggregate-cultured.

The pharmaceutical composition may further include single cells that do not generate hematospheres in the 3D aggregate culturing.

The hematospheres or the single cells may include mononcyte cells, anti-inflammatory type 2 T lymphocytes cells, regulatory lymphocytes cells, and the like.

The immune-related diseases may include autoimmune diseases and chronic inflammatory diseases.

The present invention also provides a method of treating immune-related diseases by administering a pharmaceutically effective dose of the pharmaceutical composition to a subject.

The present invention provides a pharmaceutical composition for treating ischemic diseases, containing blood-born hematospheres.

The blood-born hematospheres may be generated when mononcyte cells are isolated from human blood and then 3D aggregate-cultured.

The blood-born hematospheres may be isolated into single cells and then used.

The blood-born hematospheres may be induced to vascular endothelial cells and vascular smooth muscle cells.

The blood-born hematospheres may form a blood vessel.

The ischemic diseases may include ischemic cardiac diseases, myocardial infarction, angina pectoris, limb ischemia, and the like.

The present invention also provides a method of treating ischemic diseases by administering a pharmaceutically effective dose of the pharmaceutical composition to a subject.

The present invention provides a pharmaceutical composition for promoting lymphatic neovascularization, containing blood-born hematospheres.

The blood-born hematospheres may be generated when mononcyte cells are isolated from human blood and then 3D aggregate-cultured.

The blood-born hematospheres may be isolated into single cells and then used.

The blood-born hematospheres may include lymphatic vessel adult stem cells and progenitor cells.

The pharmaceutical composition may further include platelets.

The pharmaceutical composition may be used for healing wounds.

The pharmaceutical composition may be used for treating diseases including lymphatic dysplasia or other lymphatic disorders.

The present invention also provides a method of treating diseases having lymphatic dysplasia or other lymphatic disorders by administering the pharmaceutical composition to a subject.

The present invention provides a pharmaceutical composition for treating neurological diseases, containing blood-born hematospheres.

The blood-born hematospheres may be generated when monocyte cells are isolated from human blood and then 3D aggregate-cultured.

After the blood-born hematospheres are generated, when the medium is changed to a medium promoting differentiation into nerve cells, neural progenitor cells and/or nerve cells may be induced.

The neurological diseases may include neurodegenerative diseases, ischemic neurological diseases, nerve injury diseases, and the like.

The present invention also provides a method of treating neurological diseases by administering a pharmaceutically effective dose of the pharmaceutical composition to a subject.

In order to address the above-described objects, the present invention provides a pharmaceutical composition for treating metabolic diseases, containing blood-born hematospheres.

The blood-born hematospheres may be generated when monocyte cells are isolated from human blood and then 3D aggregate-cultured.

The blood-born hematospheres may be induced to insulin secreting cells.

The metabolic diseases may be selected from the group consisting of diabetes, hyperlipidemia, and obesity.

The present invention also provides a method of treating metabolic diseases by administering a pharmaceutically effective dose of the pharmaceutical composition according to the present invention to a subject.

### Advantageous Effects

Blood-born hematospheres prepared by isolating monocytes from human blood have a differentiation capacity to differentiate into cells of different systems in a specific environment, are more smoothly supplied than other stem cell sources since a source thereof is human blood, and have a very low isolation cost. In addition, since there are no risks of immunity and teratomas, there are big advantages in practice of clinical applications. The blood-born hematospheres are autologous adult stem cells having well-proven stability that may have the effect of an operation on a patient with no surgical operation, and have rich extracellular matrixes, cytokines, growth factors, and the like.

According to the present invention, when hematospheres are prepared by 3D aggregate-culturing monocyete cells in blood, anti-inflammatory monocytes, type 2 T lymphocytes, and regulatory lymphocytes may be generated and amplified inside and outside hematospheres. These are derived from humans rather than rodents and may be produced in vitro. Therefore, problems and inconvenience in that study directly connected with a disease model, which have been recently difficult may be solved. Also, ethical issues may be solved. In addition, according to the present invention, it is possible to solve previous problems in that only small amounts of human blood-derived anti-inflammatory monocyte cells, type 2 helper lymphocytes, and regulatory lymphocytes are collected by administering specific cytokines and other biochemical materials. In addition, since anti-inflammatory monocytes, type 2 T lymphocytes, and regulatory lymphocytes according to the present invention are patient-derived immune cells, they may also be effectively used for cell treatment of various immune-related diseases including autoimmune diseases. Eventually, blood-born hematospheres of the present invention including large amounts of anti-inflammatory monocytes, type 2 T lymphocytes, or regulatory lymphocytes are expected to be used for development of a cell therapeutic agent for various immune-related diseases including cancer.

Also, by inducing vascular progenitor cells and vascular smooth muscle cells and enabling angiogenesis, blood-born hematospheres are expected to be eventually used for development of a cell therapeutic agent that may treat ischemia-related diseases.

In addition, extensive amplification of lymphatic vessel adult stem cells and progenitor cells having a lymphatic vessel marker, which are present in blood in small amounts, is possible and thereby potency of stem cells may be maximized.

Therefore, the present invention is expected to contribute to development of an optimal autologous cell therapeutic agent for cell treatment of lymphatic vessel-related diseases.

Also, when the blood-born hematospheres are used, it is possible to amplify vascular progenitor cells, and differentiate into vascular endothelial cell and vascular smooth muscle cells. Therefore, it may contribute to developing a therapeutic agent for ischemic diseases.

In addition, the blood-born hematospheres are used to induce neural progenitor cells and/or nerve cells, and development of a cell therapeutic agent capable of treating nerve injury-related diseases is expected.

Also, according to the present invention, differentiation of insulin secreting cells using blood-born hematospheres is possible. When the insulin secreting cells are used, it is expected to contribute to practical development of a cell therapeutic agent for metabolic diseases.

When the blood-born hematospheres according to the present invention are used, existing problems in development of a stem cell therapeutic agent, that is, tumor occurrence, immune rejection, ethical issues, differentiation methods, a difficulty in obtaining existing cells, a high cost, and the like, may be addressed.

### DESCRIPTION OF DRAWINGS

FIG. 1 is a diagram schematically illustrating a process in which monocyte cells isolated from human blood are aggregated and cultured at a high density using a 3D culturing method, and then blood-born hematospheres (BBHSs) are cultured and prepared.

FIG. 2 shows diagrams illustrating changes in anti-inflammatory monocytes in peripheral blood mononuclear cells (PBMCs) and blood-born hematospheres measured by a FACS technique.

FIG. 3 is a diagram illustrating the results obtained by performing reverse transcriptase-polymerase chain reaction (RT-PCR) in order to determine expression of anti-inflammatory-related genes in blood-born hematospheres.

FIG. 4 shows diagrams illustrating the results obtained by comparing expression of anti-inflammatory and inflammatory cytokines when blood-born hematospheres are 3D-cultured and peripheral blood mononuclear cells (PBMCs) are 2D-cultured.

FIG. 5 shows diagrams schematically illustrating blood-born hematospheres when isolated monocyter cells are 3D-cultured, blood-born hematospheres and single cells (non-BBHSs) isolated therefrom are generated.

FIG. 6 shows diagrams illustrating changes in type 1, type 2, and type 17 helper lymphocytes, and regulatory lymphocytes when peripheral blood mononuclear cells and blood-born hematospheres are 3D-cultured.

FIG. 7 is a graph showing absolute counts of type 1, type 2, and type 17 helper lymphocytes, and regulatory lymphocytes when blood-born hematospheres are cultured for 3 days and 5 days.

FIG. 8 shows the results obtained by determining regulatory lymphocytes (CD4(+)/Foxp3(+)) in single cells (non-BBHSs) isolated from blood-born hematospheres using an immunofluorescence assay.

FIG. 9 shows the results obtained by analyzing helper and regulatory lymphocytes-related anti-inflammatory and inflammatory cytokines using an enzyme-linked immunosorbent assay (ELISA) after blood-born hematospheres are cultured.

FIG. 10 illustrates the results showing a possibility of self-sprouting when blood-born hematospheres are cultured in a Matrigel-coated dish.

FIG. 11 illustrates the results showing expression of vascular endothelial growth factor receptors 2 (VEGFR-2, KDR, red, arrow) when blood-born hematospheres are cultured in a Matrigel-coated dish.

FIG. 12 illustrates the results showing expression of vascular endothelial growth factor receptors 2 (VEGFR-2, KDR green) and platelet endothelial cell adhesion molecules (PECAM-1, red) and observation of Tip cells (arrow) when blood-born hematospheres are cultured in a Matrigel-coated dish.

FIG. 13 illustrates the results showing expression of vascular endothelial growth factor (VEGF, green, top) and C-X-C chemokine receptor type 4 (CXCR4, green, bottom) in blood-born hematospheres determined by an immunofluorescence assay.

FIG. 14 illustrates the results showing a significant decrease in generation of blood-born hematospheres when VEGF and VEGFR2 (KDR) are suppressed using VEGF antibodies and a chemical inhibitor (SU1498) of VEGFR2 (KDR) which is a receptor thereof.

FIG. 15 illustrates the results showing expression of cytokines and receptors thereof which are known to be important in angiogenesis in blood-born hematospheres determined by RT-PCR and ELISA.

FIG. 16 illustrates the results showing an increased activity of matrix metallopeptidase 9 (MMP-9) using supernatants of blood-born hematospheres determined by an MMP-9 Zymography assay.

FIG. 17 illustrates the results showing an increased migration and tube formation of HUVEC when human umbilical vein endothelial cells (HUVECs) are cultured using supernatants of blood-born hematospheres, including micrographs (top) and quantitative graphs (bottom).

FIG. 18 shows the results when ischemia is induced in a hindlimb of a nude mouse having a degraded immune system, blood-born hematospheres are injected, perfusion is measured by laser Doppler perfusion imaging (LDPI), and an immunofluorescence assay was performed using antibodies specific to human cells of cluster of differentiation 34 (CD34, green) serving as a vascular endothelial cell marker and alpha smooth muscle actin (SMA-α, red).

FIG. 19 is a diagram schematically illustrating a process in which monocyte cells isolated from human blood are aggregate-cultured at a high density by a 3D culturing method and then blood-born hematospheres (BBHSs) are cultured and prepared.

FIG. 20 shows the results obtained by determining expression of podoplanin serving as a lymphatic vessel-related marker in blood-born hematospheres over time by flow cytometry.

FIG. 21 shows the results obtained by determining expression of podoplanin proteins and VEGFR3 proteins which are lymphatic vessel-related markers in blood-born hematospheres over time by Western blot.

FIG. 22 shows the results obtained by determining expression of podoplanin and VEGFR3 which are lymphatic vessel-related markers in blood-born hematospheres by an immunofluorescence assay.

FIG. 23 shows the results obtained by determining gene changes of lymphatic vessel-related markers in blood-born hematospheres over time by polymerase chain reaction.

FIG. 24 shows the results obtained by determining gene changes of lymphatic vessel-related markers of positive cells and negative cells by real time polymerase chain reaction after podoplanin which is a representative lymphatic vessel marker is isolated from blood-born hematospheres.

FIG. 25 shows a healing effect of blood-born hematospheres and platelets through a wound healing model of immune-deficient mice.

FIG. 26 shows sections of a back of the immune-deficient mice which is immune-stained with a lymphatic vessel marker.

FIG. 27 shows sections of an ear of the immune-deficient mice which is immune-stained with a lymphatic vessel marker.

FIG. 28 is a diagram schematically illustrating a process in which blood-born hematospheres are generated and then induced to differentiate into nerve cells.

FIG. 29 shows images of cells after differentiation into nerve cells is induced in the same way as in the schematic diagram in FIG. 28.

FIG. 30 shows the results obtained by determining whether nerve cell differentiation is induced and then whether neural progenitor cells are induced by an immunofluorescence assay (green indicates Nestin and red indicates Musashi).

FIG. 31 shows the results obtained by determining whether nerve cell differentiation is induced and then whether nerve cells are induced by an immunofluorescence assay (green indicates Sox2 and red indicates beta-III tubulin).

FIG. 32 shows the result obtained by determining whether some cells express insulin in blood-born hematospheres by an immunofluorescence assay (green: insulin and blue: nucleus).

FIG. 33 shows the result obtained by determining expression of Nestin in blood-born hematospheres (BBHSs) by an immunofluorescence assay (green: Nestin, blue: nucleus, and Scale bar: 50 um).

FIG. 34 shows the result obtained by determining expression of beta-tubulin III in blood-born hematospheres (BBHSs) by an immunofluorescence assay (green: beta-tubulin III, blue: nucleus, and Scale bar: 50 um).

FIG. 35 is a diagram illustrating a process in which blood-born hematospheres are induced to differentiate into insulin secreting cells.

FIG. 36 shows the results that expression increases to a fourth step when genes important for insulin expression are determined using RT-PCR in each step of insulin secreting cell differentiation induction.

FIG. 37 shows the results that most cells express insulin and some cells express Nestin when blood-born hematospheres (BBHSs) differentiate into insulin secreting cells and then insulin (red) and Nestin (green) are stained by an immunofluorescence assay (red: insulin, green: Nestin, blue: nucleus, and Scale bar: 50 um).

FIG. 38 shows the results that the greatest amount of red is observed in the final fourth step of insulin secreting cell differentiation when a Dithizone staining method in which insulin is detected and stained with red is performed on blood-born hematospheres and each step of insulin secreting cell differentiation (Scale bar: 10 um).

FIG. 39 shows the results that insulin is secreted in high glucose when glucose-stimulated insulin secretion (GSIS) of insulin secreting cells induced from blood-born hematospheres (BBHSs) is compared.

### MODES OF THE INVENTION

The present invention provides a novel method of amplifying anti-inflammatory monocytes, type 2 T lymphocytes, or regulatory lymphocytes in vitro using blood-born hematospheres and a method of treating immune-related diseases using the blood-born hematospheres.

Also, the present invention provides a pharmaceutical composition for treating ischemic diseases containing blood-born hematospheres. The ischemic diseases include ischemic cardiac disease, myocardial infarction, angina pectoris, limb ischemia, and the like.

Furthermore, the present invention provides a method of effectively extensively culturing and proliferating lymphatic vessel adult stem cells and progenitor cells which are present in blood in small amounts using blood monocyte cells and a method of treating diseases having lymphatic dysplasia and other lymphatic disorders using the same.

Moreover, the present invention provides a pharmaceutical composition for treating neurological diseases, containing blood-born hematospheres.

In addition, the present invention provides a pharmaceutical composition for treating metabolic diseases containing blood-born hematospheres. The blood-born hematospheres are induced to insulin secreting cells. The metabolic diseases include diabetes, hyperlipidemia, obesity, and the like, but the metabolic diseases are not limited thereto, as long as diseases are caused in association with an insulin secreting metabolic function.

The term “blood-born hematospheres (BBHSs)” used in the present invention refers to an aggregate of monocyte cells in blood and specific cells having stemness which forms a 3D structure and forms a spherical shape as in inner cell masses of blastocysts.

The term “anti-inflammatory monocyte cells” used in the present invention refers to monocyte cells having anti-inflammatory properties similar to myeloid-derived suppressor cells (MDSCs) which have been recently known by immunologists as cells other than monocyte cells having original immunity. The monocyte cells refer to anti-inflammatory monocyte cells that may facilitate vasculogenesis as cells other than monocyte cells originally having high immunity.

The term “type 2 T lymphocytes (Th2)” or “regulatory T lymphocytes (Treg)” used in the present invention refers to important lymphocytes, indispensable to an adaptive immune system. These indirectly influence the immune system and kill host cells infected with pathogens or viruses to prevent further propagation thereof.

The present invention provides a pharmaceutical composition for treating immune-related diseases containing blood-born hematospheres. The hematospheres may include anti-inflammatory monocyte cells, type 2 T lymphocytes, or regulatory lymphocytes. Also, the immune-related diseases include various types of autoimmune diseases and chronic inflammatory diseases.

The pharmaceutical composition of the present invention may include a pharmaceutically acceptable carrier. The pharmaceutically acceptable carrier may include a normal saline, a polyethylene glycol, ethanol, a vegetable oil, and/or isopropyl myristate, and the like, but the carrier is not limited thereto.

Also, the present invention provides a method of treating immune diseases by administering a pharmaceutically effective dose of the pharmaceutical composition to a subject. The term “subject” in the present invention refers to a target needing treatment of diseases, and more specifically, mammals such as humans or non-human apes, mice, rats, dogs, cats, horses, and cows. Also, in the present invention, it is apparent to those skilled in the art that a range of “pharmaceutically effective dose” is variously adjusted depending on a patient's body weight, age, gender, health condition, diet, administration time, administration method, excretion rate, severity of the disease, and the like.

A preferred dose of the pharmaceutical composition of the present invention varies depending on a patient's condition, body weight, degree of disease, drug form, administration route, and duration, but it may be appropriately selected by those skilled in the art. However, administration is performed for a day, preferably, 0.001 to 100 mg/body weight (kg), and more preferably, 0.01 to 30 mg/body weight (kg). Administration may be performed once a day or may be divided into several times.

The pharmaceutical composition of the present invention may be administered to mammals such as a rat, mouse, livestock, and human via various routes. The administration method is not limited, and administration may be performed, for example, by oral, rectal, or intravenous, intramuscular, subcutaneous, intrauterine subdural, or intracerebroventricular injections.

The term “lymphatic vessel” used in the present invention refers to a lymphatic vessel that is constituted by lymphatic endothelial cells, absorbs an interstitial fluid, proteins, fats, and the like, sends these back to a system, and performs an important role for maintaining homeostasis in tissues and immune surveillance.

The term “podoplanin” used in the present invention refers to a type-1 integral membrane glycoprotein and is known to be typically expressed in lymphatic endothelial cells.

The term “vascular endothelial growth factor receptor 3 (VEGFR3)” used in the present invention refers to a tyrosine kinase receptor of a vascular endothelial growth factor-C/D (VEGF-C/D) and is associated with lymphangiogenesis and maintenance of lymphatic endothelial cells.

The inventors generated a nerve cell friendly microenvironment through blood-born hematospheres (BBHSs) and investigated a differentiation capacity of monocyte cells into nerve cells. The nerve cell friendly microenvironment generated through blood-born hematospheres exhibits an anti-apoptotic effect in neural stem cells, an anti-apoptotic effect in differentiated nerve cell lines, an effect of inducing differentiation from neural stem cells into nerve cells, a central nerve system regeneration effect, a peripheral nerve regeneration effect, and the like. The present invention has an effect of promoting survival and differentiation of nerve cells in stem cell therapy and gene therapy using blood-born hematospheres, and is expected to be used for development of a stem cell therapeutic agent for preventing various types of neurodegenerative diseases such as dementia and cerebral ischemia, other ischemic neurological diseases, or nerve injury diseases.

Therefore, the present invention provides a pharmaceutical composition for treating neurological diseases containing blood-born hematospheres. The blood-born hematospheres are induced to neural progenitor cells, nerve cells, and the like. The neurological diseases include neurodegenerative diseases, ischemic neurological diseases, nerve injury diseases, and the like.

In addition, the inventors generated an insulin secreting cell friendly microenvironment through blood-born hematospheres (BBHSs) and investigated a differentiation capacity of monocyte cells into insulin secreting cells. The present invention has an effect of promoting survival and differentiation of insulin secreting cells in stem cell therapy and gene therapy using blood-born hematospheres, and is expected to be used for development of a stem cell therapeutic agent for various types of metabolic diseases such as diabetes, hyperlipidemia, and obesity.

Therefore, the present invention provides a pharmaceutical composition for treating metabolic diseases containing blood-born hematospheres. The blood-born hematospheres are induced to insulin secreting cells. The metabolic diseases include diabetes, hyperlipidemia, obesity, and the like but the metabolic diseases are not limited thereto, as long as diseases are caused in association with an insulin secreting metabolic function.

Hereinafter, the present invention will be described in greater detail with reference to the following Examples. However, the following Examples are provided to easily understand the present invention and the scope of the present invention is not limited to the following Examples.

### Example 1

### Example <1.1>

### Generation of Blood-Born Hematospheres (BBHSs)

(1) Step of Isolating Monocyte Cells from Peripheral Blood

Peripheral blood was obtained using a heparin (about 100 ul)-coated 50 ml syringe. 10 ml of the peripheral blood was input to a 50 ml tube. 30 ml of a phosphate-buffered saline (PBS) was added thereto and carefully mixed. 10 ml of Ficoll applied to the diluted blood to express a density gradient was slowly added into the bottom of the tube using a pipet aid. A transparent Ficoll layer and a red blood layer were separated and then centrifuged at 2,500 rpm and 25° C. for 30 minutes with a minimum stop rate. A yellow serum layer, a white monocyte layer, and a transparent Ficoll layer were isolated in an upper portion and a red layer of red blood cells and a polynuclear layer were isolated in a bottom portion. After isolation was confirmed, the yellow serum layer at an upper portion was taken out and then the white monocyte layer was carefully transferred to a new tube. The transferred monocyte layer was divided into two tubes, and the tubes were then filled with PBS and centrifuged at 1,800 rpm and 4° C. for 10 minutes. Cell pellets were suspended into single cells by vortexing and then the tubes were then filled with PBS and centrifuged at 1,700 rpm and 4° C. for 10 minutes. The above washing process was repeated three times to remove substances used to express the density gradient and residual substances in blood. The number of cells was measured using a hemocytometer before the final centrifugation is performed.

### (2) Step of 3D Culturing

Cells that had finished the washing process were suspended in an ultra-low attachment culture dish at a high density of 10⁶/ml or more using a culture solution in which 5% FBS was added to an endothelial basal medium-2 (EBM-2), were cultured in an incubator into which 5% CO₂ was supplied at 37° C., and the same medium was added to the culture after the first 2 days.

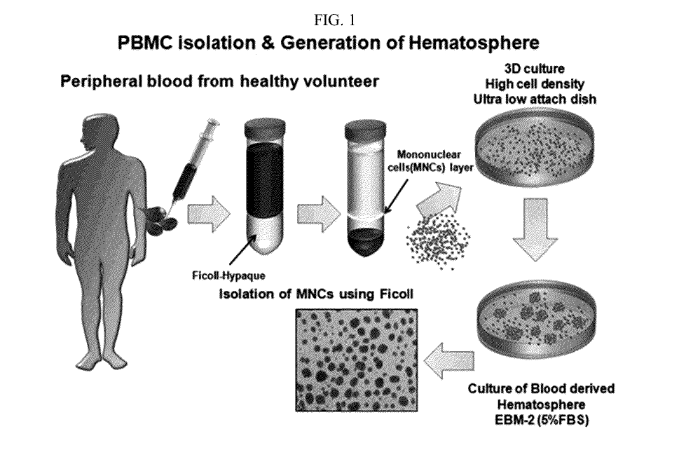

FIG. 1 is a diagram schematically illustrating a process in which peripheral blood mononuclear cells (PBMCs) are isolated and then blood-born hematospheres (BBHSs) are cultured for 5 days.

(3) Step of Dissociating Hematospheres into Single Cells

The hematospheres obtained through the culturing process underwent the process of being isolated to single cells for use in characteristic analysis and treatment. The suspended hematospheres were gathered at a center by horizontally and vertically shaking the culture dish. Only the hematospheres were transferred to a tube under a microscope and centrifuged at 1,700 rpm and 4° C. for 10 minutes. Cell pellets were gently detached using 1 ml of Accutase serving as a cell dissociation solution, and cultured in an incubator at 37° C. for 2 to 3 minutes. Cells of which incubation had finished were added with 1 ml of a cell culture solution and pipetted several times. The tubes were filled with PBS and centrifuged at 1,700 rpm and 4° C. for 10 minutes.

In the following example, an experiment in which inflammatory monocyte cells are mostly polarized to anti-inflammatory monocyte cells through generation of blood-born hematospheres (BBHSs) in Example <1.1> and an increase in type 2 helper cells or regulatory lymphocytes is determined was performed.

### Example <1.2>

### Step of Determining Increase in Anti-Inflammatory Monocytes

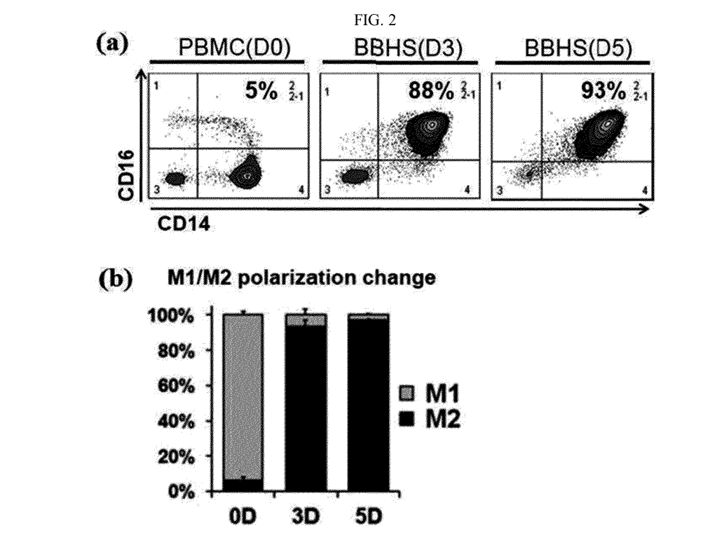

Peripheral blood mononuclear cells (PBMCs) and blood-born hematospheres (BBHSs) generated according to the present invention were cultured for 3 days and 5 days. Then, only hematospheres were selected to determine a change in anti-inflammatory monocytes through a FACS technique. In general, CD14(+)CD16(−) is known as a marker of inflammatory monocytes (M1), and CD14(+)CD16(+) is known as a marker of anti-inflammatory monocytes (M2). Inflammatory monocytes (M1) and anti-inflammatory monocytes (M2) were analyzed using the markers. FIG. 2 shows the results.

Most peripheral blood mononuclear cells initially are the inflammatory monocytes (M1). The anti-flammatory monocytes (M2) are included in a small amount of about 5%. However, as shown in FIG. 2, it can be seen that the number of anti-inflammatory monocytes significantly increased when hematospheres (BBHSs) were cultured for 3 days and 5 days. In addition, it can be seen that the number of anti-inflammatory monocytes significantly increased to about 88% at the initial culture (D3) (refer to FIG. 2A). When the experiment was performed on 8 volunteers, similar results were obtained (refer to FIG. 2B).

### Example <1.3>

### Determination Expression of Anti-Inflammatory-Related Genes and Increase in Cytokine Secretion

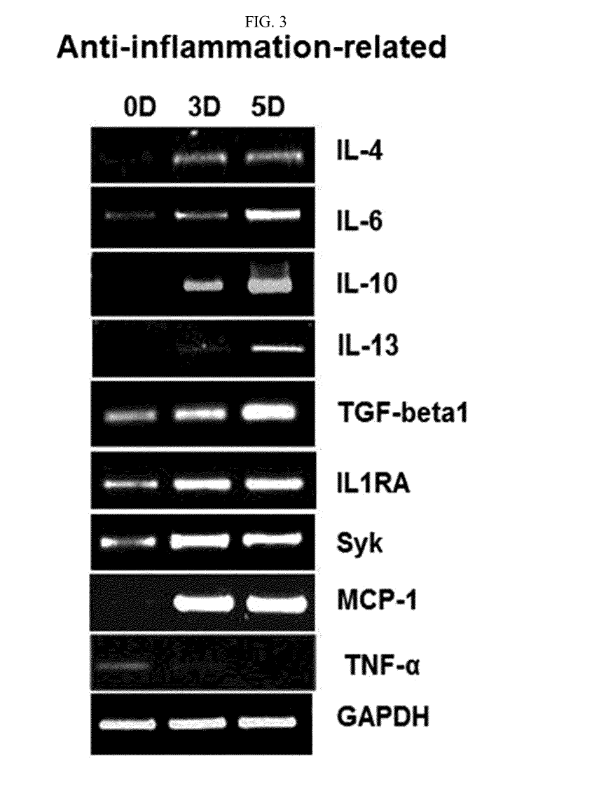

In FIG. 3, RNA was extracted from peripheral blood mononuclear cells (PBMCs) and hematospheres (BBHSs) obtained in Example <1.1>, and then RT-PCR was performed to identify expression of anti-inflammatory-related genes. FIG. 3 shows the results.

As results, as shown in FIG. 3, hematospheres (BBHSs) showed significantly increased expression of Interleukin-4 (IL-4), IL-6, IL-10, IL-13, the transforming growth factor beta 1 (TGF-beta1), IL-1RA, Syk, and MCP-1 than peripheral blood mononuclear cells (PBMCs, OD). For hematospheres, it was determined that the expression further increased when the culture was performed for 5 days (5D) than when the culture was performed for 3 days (3D).

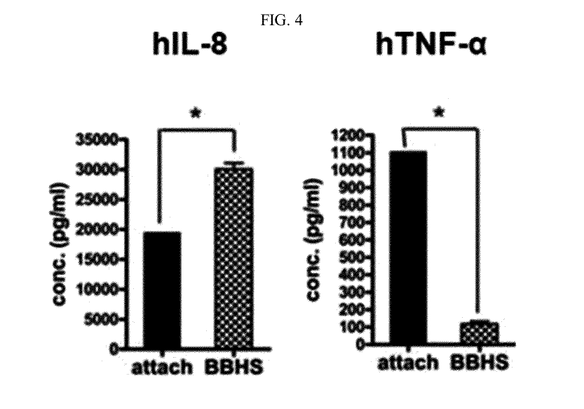

In addition, a group in which hematospheres (BBHSs) obtained in Example <1.1> and PBMCs were attached was cultured for 5 days. Specifically, supernatants of suspension (3D) culture and attached (2D) culture were obtained to compare secretion of anti-inflammatory and inflammatory cytokines. FIG. 4 shows the results.

As shown in FIG. 4, when hematospheres (BBHSs) are generated, anti-inflammatory cytokine IL-8 increased and inflammatory cytokine TNF-α decreased, compared to the group to which PBMCs were attached (Attach in FIG. 4).

### Example <1.4>

### Analysis of Type 1 T Lymphocytes, Type 2 T Lymphocytes and, Regulatory Lymphocytes

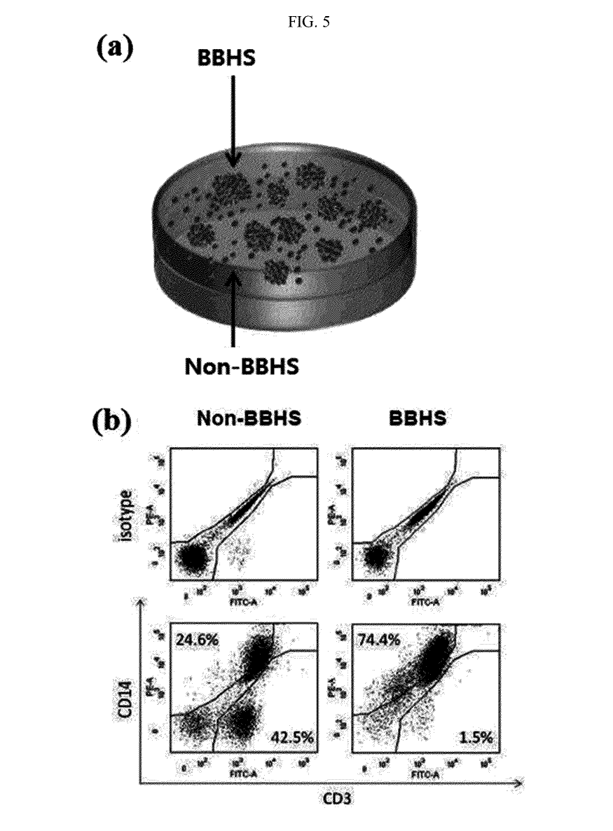

When human peripheral blood monocytes (hPBMC) were 3D-cultured to generate blood-born hematospheres (BBHSs), the monocytes are divided into a group in which hematospheres (BBHSs) are generated and a group in which no hematospheres are generated and single cells (non-BBHSs) are maintained. The following analysis was performed on cells of the above two groups (refer to FIG. 5A).

As a result, as shown in FIG. 5B, the group in which hematospheres (BBHSs) are generated was mostly formed of monocytes, CD14(+) cells. The single cell (non-BBHSs) group was mostly formed of lymphocytes, CD3(+) cells.

Specifically, human peripheral blood monocytes (hPBMCs) and hematospheres (BBHSs) were cultured for 3 days and 5 days, and then the hematospheres were removed. Then, lymphocytes in single cells (non-BBHSs, non incorporated BBHSs) were analyzed. As an analysis method, each cell was treated with 50 ng/ml of phorobol 12-myristate 13-acetate (PMA) (Sigma Aldrich) and 25 ng/ml of Ionomycin (Sigma Aldrich) to provide stimulation for 4 hours and then was treated with Monensin (BD biosciences). Then, the following antibodies were used to analyze type 1 helper lymphocytes, type 2 helper lymphocytes, and regulatory lymphocytes [CD4 FITC (BD biosciences), IFN-γPE (R&D systems), STATE APC (R&D systems), IL-4 Pe-Cy7 (BD biosciences), CD25 APC-Cy7 (BD biosciences), Foxp3 APC (eBioscience), and IL-17A FITC (eBioscience)].

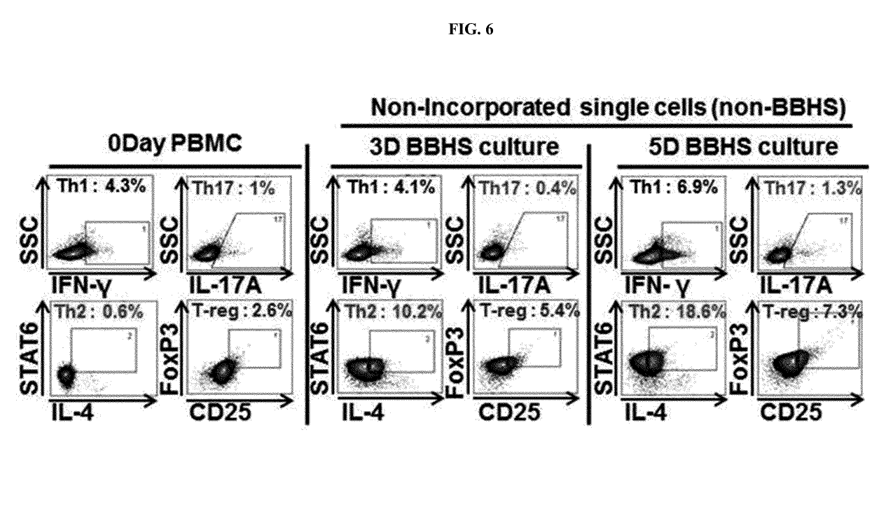

As shown in FIG. 6, compared to immediately after human peripheral blood monocytes (hPBMC) were isolated (0 day PBMC), when hematospheres (BBHSs) were cultured for 3 days and 5 days, the hematospheres were removed and then single cells (non-BBHSs, non incorporated BBHSs) were analyzed, it was determined that type 2 helper lymphocytes (CD4(+)IL-4(+)STAT6(+)) and regulatory lymphocytes (CD4(+)CD25(+)FoxP3(+)), which are important for an anti-inflammatory ability, increased over time in 3D culturing compared to fresh PBMCs. On the other hand, there was no big change in inflammatory CD4(+)IFN-gamma(+) (Th1), and CD4(+)IL-17A(+) (Th17).

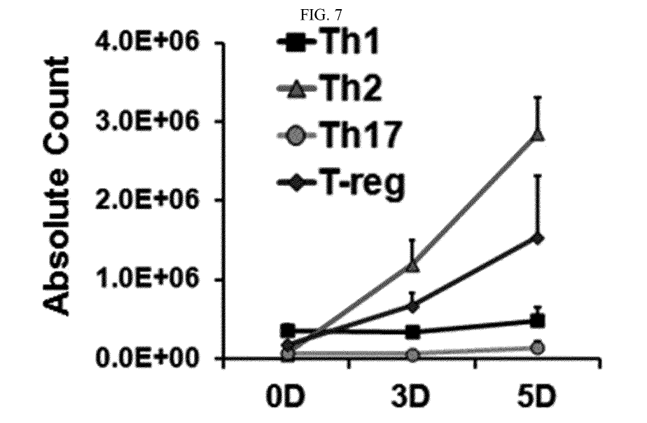

In FIG. 7 using the same method in FIG. 6, immediately after human peripheral blood monocytes (hPBMC) were isolated (0 day PBMC), hematospheres (BBHSs) were cultured for 3 days and 5 days, and then the hematospheres were removed. In single cells (non-BBHSs, non incorporated BBHSs), the absolute number of lymphocytes was measured in type 2 helper lymphocytes (CD4(+)IL-4(+)STAT6(+)), regulatory lymphocytes (CD4(+)CD25(+)FoxP3(+)), type 1 helper lymphocytes (CD4(+)IFN-Gamma(+)), and type 17 helper lymphocytes (CD4(+)IL-17A(+)). FIG. 7 shows the results. As shown in the graph, the number of cells increased in anti-inflammatory type 2 helper lymphocytes and regulatory lymphocytes. However, there was no change in the number of cells in inflammatory type 1 helper lymphocytes and type 17 helper lymphocytes.

Also, hematospheres (BBHSs) were cultured for 5 days, and the hematospheres were removed. Then, the same experiment as described above was performed on single cells (non-BBHSs, non incorporated BBHSs). By an immunofluorescent (IF) technique, CD4/Foxp3, which is a marker of regulatory lymphocytes, was stained. FIG. 8 shows the results.

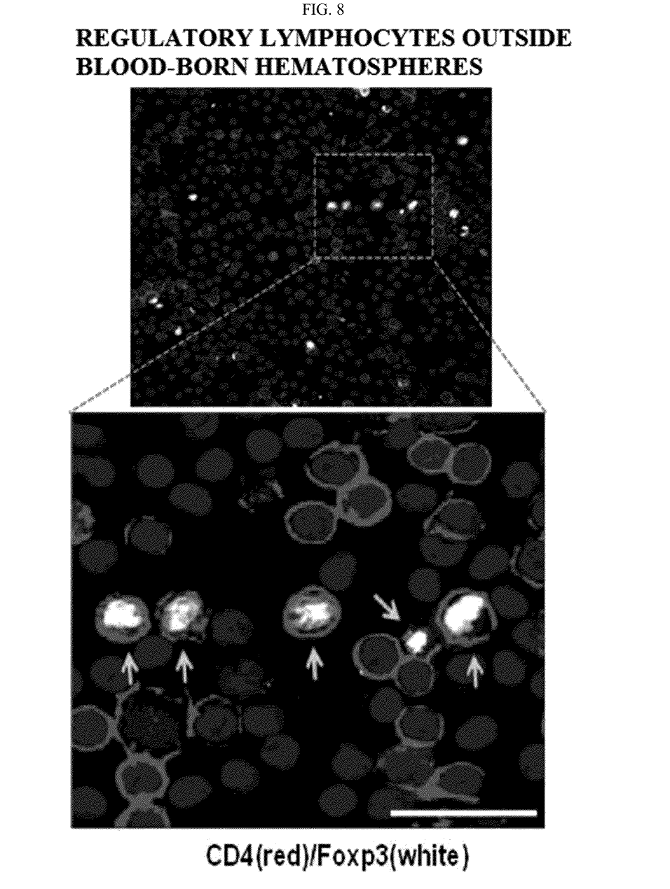

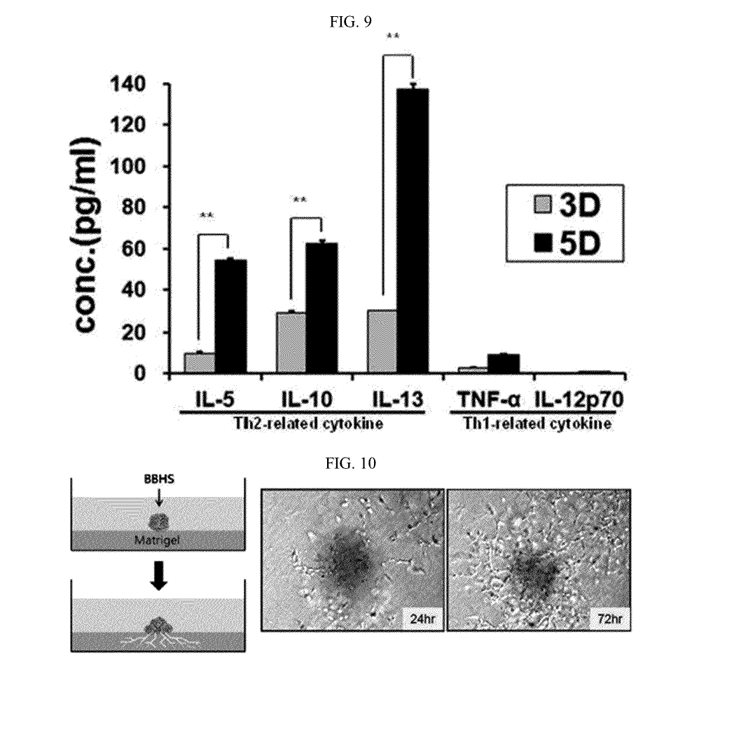

In addition, blood-born hematospheres (BBHSs) were cultured for 3 days and 5 days and then supernatants were obtained to perform an enzyme-linked immunosorbent assay (ELISA), which was then performed. FIG. 9 shows the results.

As a result, as shown in FIG. 9, when the culture was performed for 3 days and 5 days, inflammatory lymphocytes (type 1 lymphocytes-related cytokines (TNF-α and IL-12p70)) rarely appeared. On the other hand, it can be seen that anti-inflammatory lymphocytes (type 2 lymphocytes-related cytokines (IL-5, IL-10, and IL-13)) appeared even when the culture was performed for 3 days, and were further secreted when the culture was performed for 5 days.

From the above result, according to the method of the present invention, since it is possible to effectively produce anti-inflammatory monocytes, type 2 T lymphocytes, or regulatory lymphocytes without using specific cytokines, the method may be expected to contribute development of a cell therapeutic agent for autoimmune diseases, chronic inflammatory diseases, and the like.

### Example 2

### Example <2.1>

### Generation of Blood-Born Hematospheres (BBHSs)

(1) Step of Isolating Monocyte Cells from Peripheral Blood

Peripheral blood was obtained using a heparin (about 100 ul)-coated 50 ml syringe. 10 ml of the peripheral blood was input to a 50 ml tube. 30 ml of a phosphate-buffered saline (PBS) was added thereto and carefully mixed. 10 ml of Ficoll applied to the diluted blood to express a density gradient was slowly added into the bottom of the tube using a pipet aid. A transparent Ficoll layer and a red blood layer were separated and then centrifuged at 2,500 rpm and 25° C. for 30 minutes with a minimum stop rate. A yellow serum layer, a white mononcyte layer, and a transparent Ficoll layer were isolated in an upper portion and a red layer of red blood cells and a polynuclear layer were isolated in a bottom portion. After isolation was confirmed, the yellow serum layer at an upper portion was taken out and then the white monocyte layer was carefully transferred to a new tube. The transferred monocyte layer was divided into two tubes, and the tubes were then filled with PBS and centrifuged at 1,800 rpm and 4° C. for 10 minutes. Cell pellets were suspended into single cells by vortexing and then the tubes were filled with PBS and centrifuged at 1,700 rpm and 4° C. for 10 minutes. The above washing process was repeated three times to remove substances used to express the density gradient and residual substances in blood. The number of cells was measured using a hemocytometer before the final centrifugation is performed.

### (2) Step of 3D Culturing

Cells that had finished the washing process were suspended in an ultra-low attachment culture dish at a high density of 10⁶/ml or more using a culture solution in which 5% FBS was added to an endothelial basal medium-2 (EBM-2), and cultured in an incubator to which 5% CO₂ was supplied at 37° C., and the same medium was added to the culture after the first 2 days.

FIG. 1 is a diagram schematically illustrating a process in which peripheral blood mononuclear cells (PBMCs) are isolated and then blood-born hematospheres (BBHSs) are cultured for 5 days.

(3) Step of Dissociating Hematospheres into Single Cells

The hematospheres obtained through the culturing process underwent the process of being isolated to single cells for use in characteristic analysis and treatment. The suspended hematospheres were gathered at a center by horizontally and vertically shaking the culture dish. Only the hematospheres were transferred to a tube under a microscope and centrifuged at 1,700 rpm and 4° C. for 10 minutes. Cell pellets were gently detached using 1 ml of Accutase serving as a cell dissociation solution, and cultured in an incubator at 37° C. for 2 to 3 minutes. Cells of which incubation had finished were added with 1 ml of a cell culture solution and pipetted several times. The tubes were filled with PBS and centrifuged at 1,700 rpm and 4° C. for 10 minutes.

### Example <2.2>

### Investigation of Expression of Angiogenesis-Related Genes and Marker (In Vitro Experiment)

Expression of angiogenesis-related genes, receptors, and markers were investigated in the hematospheres (BBHSs) generated in Example <2.1>. FIGS. 10 to 17 show the results.

### (1) Determination of Self-Sprouting of Blood-Born Hematospheres (BBHSs)

A 35 mm confocal dish (ibidi) was thickly coated with about 200 ul of GFR Matrigel (BD Biosciences) on ice and then incubated in an incubator at 37° C. Then, only Sphere was isolated from blood-born hematospheres (BBHSs) cultured for 5 days under a microscope and carefully plated onto the Matrigel-thickly coated confocal dish of 35 mm. A culture solution was replaced by EGM-2MV (Lonza). After 24 hours and 72 hours have passed, sprouting of blood-born hematospheres (BBHSs) was observed under a microscope. As a result, it was determined that blood-born hematospheres (BBHSs) had self-sprouted (refer to FIG. 10).

### (2) Determination of Expression and Self-Sprouting of VEGFR2 and PECAM-1

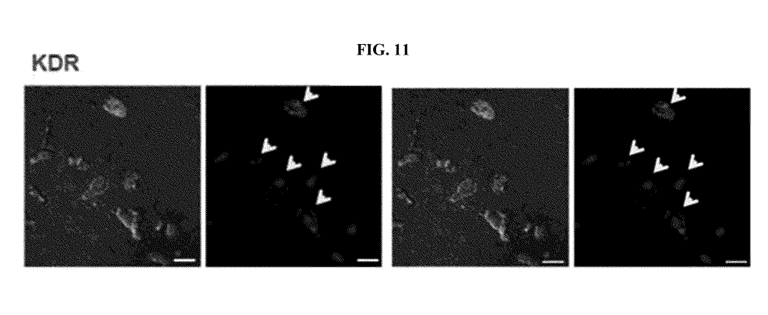

Using the same method in Example <2.2>, blood-born hematospheres were put in a thick GFR Matrigel-coated confocal dish of 35 mm, cultured for 24 hours, fixed and then washed with PBS three times. Then, blocking was performed for 30 minutes and an immunofluorescent technique was performed to localize vascular endothelial growth factor receptors 2 (VEGFR-2, KDR). As a result, as shown in FIG. 11, expression of VEGFR-2 (red, arrow) was determined.

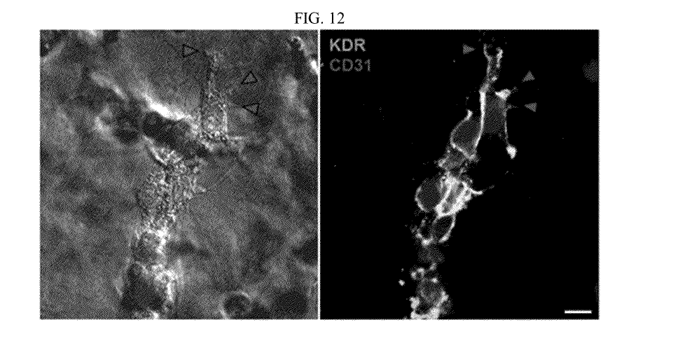

Also, when the blood-born hematospheres were cultured in a Matrigel-coated dish, as shown in FIG. 12, it was determined that not only the vascular endothelial growth factor receptors 2 (VEGFR-2, KDR green) but also platelet endothelial cell adhesion molecules (PECAM-1, red) were expressed, and tip cells (arrow) were observed.

### (3) Determination of Expression of VEGF and CXCR4

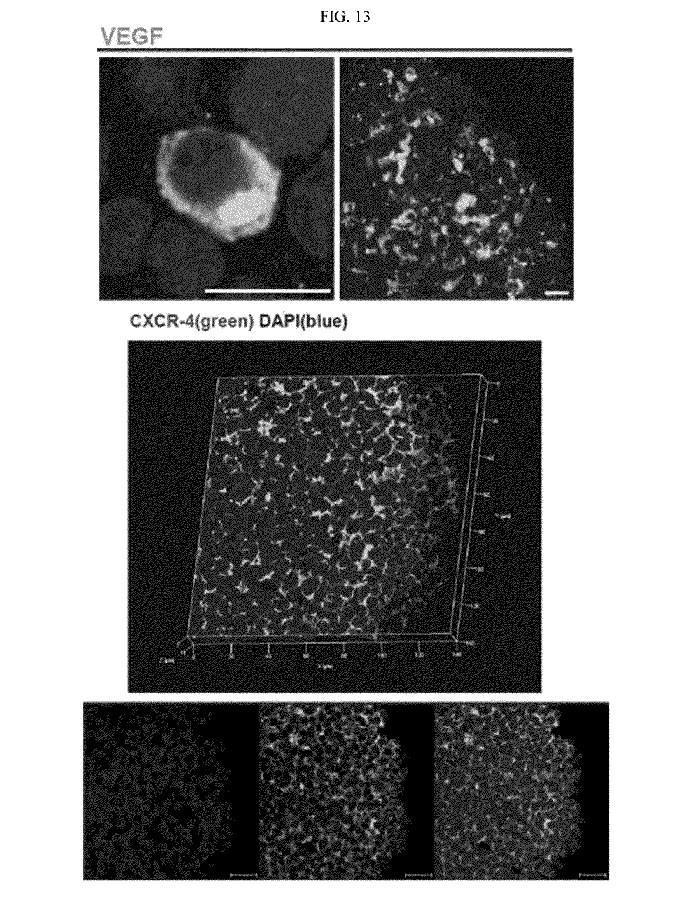

The blood-born hematospheres (BBHSs) were cultured for 5 days. Then, as shown in FIG. 13, through the immunofluorescent technique, expression of vascular endothelial growth factors (VEGF, green, top) and C-X-C chemokine receptor type 4 (CXCR4, green, bottom) was determined.

### (4) Determination of Importance of VEGF-VEGFR2 (KDR) Loop Signaling in BBHS-Angiogenic Niche

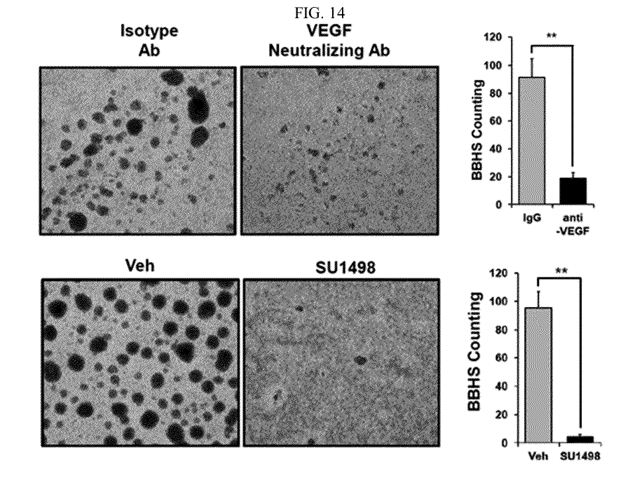

Before hematospheres are generated (0 hrs), VEGF and VEGFR2 (KDR) were suppressed using VEGF antibodies and a chemical inhibitor (SU1498) of VEGFR2 which is a receptor thereof 10 ug/ml of VEGF neutralizing antibody (R&D) and 10 uM of SU1498 (Calbiochem) which is a chemical inhibitor of VEGFR2 were also added to the treatment. As a result, as shown in FIG. 14, it was determined that generation of blood-born hematospheres (BBHSs) significantly decreased.

### (5) Determination of Expression of Cytokine and Receptor Important for Vasculogenesis Using RT-PCR

RNA was isolated from fresh Human PBMC and blood-born hematospheres (BBHSs) on the 3^{rd} day and 5th day after culturing to perform reverse transcriptase-polymerase chain reaction (RT-PCR). Amounts of IL-9, C-X-C chemokine receptor type 1 (CXCR1), CXCR2, VEGF, KDR, Hepatocyte growth factor (HGF), c-Met, Matrix metalloproteinases 9 (MMP-9), which are known to be important in angiogenesis, were determined Primers used in this case are shown in Table. 1.

TABLE 1

| | | SEQ | | | Product |
|---|---|---|---|---|---|
| | | ID | | | size |
| Primer | Sequence | NO | TM | Access Number | (bp) |
| IL-8 | Forward | 1 | 60° C. | NM_000584.3 | 178 |
| | 5′-GGCCGTGGCTCTCTTGGCAG-3′ | | | | |
| | Reverse | 2 | | | |
| | 5′-TGTGTTGGCGCAGTGTGGTCC-3′ | | | | |
| CXC | Forward | 3 | 60° C. | NM_000634.2 | 222 |
| R1 | 5′-GAGCCCCCGAATCTGACATTA-3′ | | | | |
| CXC | Reverse | 4 | | NM_000634.2 | 222 |
| R1 | 5′-AGTGCCTGCCTCAATGTCTCC-3′ | | | | |
| VEG | Forward | 5 | 60° C. | NM_001025366.2 | 211 |
| F | 5′-GGGCAGAATCATCACGAAGT-3′ | | | | |
| VEG | Reverse | 6 | | NM_001025366.2 | 211 |
| F | 5′-TGGTGATGTTGGACTCCTCA-3′ | | | | |
| KDR | Forward | 7 | 64° C | NM_002253.2 | 289 |
| | 5′-ATGCTGGACTGCTGGCACGG-3′ | | | | |
| | Reverse | 8 | | | |
| | 5′-TCACAGGCCGGCTCTTTCGC-3′ | | | | |
| HGF | Forward | 9 | 60° C. | NM_00601.4 | 168 |
| | 5′-CTGGTTCCCCTTCAATAGCA-3′ | | | | |
| HGF | Reverse | 10 | | NM_00601.4 | 168 |
| | 5′-CTCCAGGGCTGACATTTGAT-3′ | | | | |
| c- | Forward | 11 | 59° C. | NM_001127500.1 | 199 |
| Met | 5′-CCAATGGCCTGCAGCCGTGA-3′ | | | | |
| | Reverse | 12 | | | |
| | 5′-CTGTTCTGGGGCTGCCGCTC-3′ | | | | |
| MM | Forward | 13 | 56° C. | NM_004994.2 | 482 |
| P-9 | 5′-CAACATCACCTATTGGATCC-3′ | | | | |
| MM | Reverse | 14 | | NM_004994.2 | 482 |
| P-9 | 5′-CGGGTGTAGAGTCTCTCGCT-3′ | | | | |
| GAP | Forward | 15 | 60° C. | NM_002046.3 | 185 |
| DH | 5′-GAGTCAACGGATTTGGTCGT-3′ | | | | |
| | Reverse | 16 | | | |
| | 5′-GACAAGCTTCCCGTTCTCAG-3′ | | | | |

As shown in FIG. 15B, compared to fresh Human PBMC (OD), when BBHSs was cultured for 3 days (3D) and 5 days (5D), the cytokines known to be important in angiogenesis increased.

Also, as shown in FIG. 15B, in order to determine whether molecules known to be important in angiogenesis were actually secreted in addition to RNA, ELISA was performed. As a result, it was determined that secretion of VEGF, HGF, and IL-8 increased in hematospheres (BBHSs) compared to attached PBMCs.

### (6) Determination of Increase in Activity of MMP-9

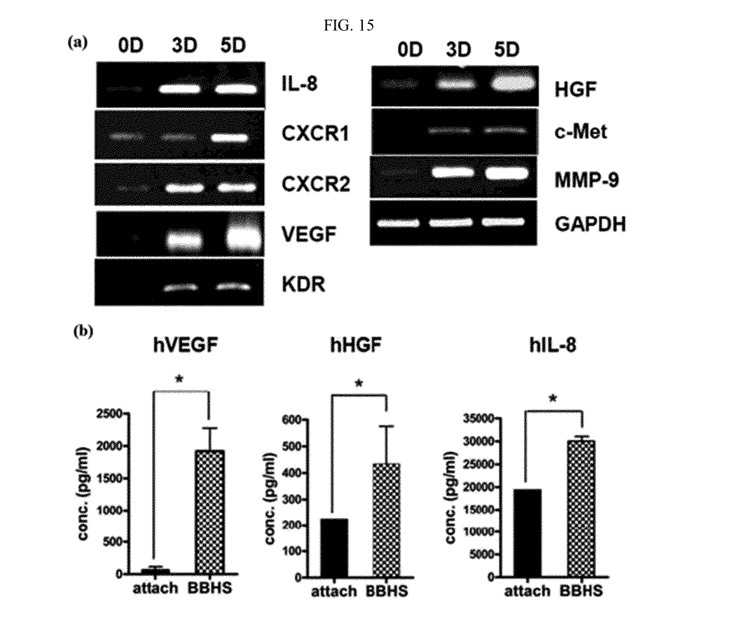

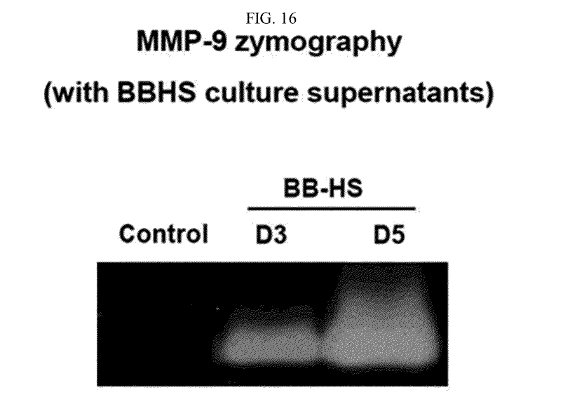

An MMP-9 Zymography assay was performed using supernatants of blood-born hematospheres. As shown in FIG. 16, it was determined that an activity of Matrix metallopeptidase 9 (MMP-9) increased.

(7) Determination of increase in migration and tube formation of HUVEC

When supernatants of blood-born hematospheres (BBHSs) cultured for 5 days were used to culture human umbilical vein endothelial cells (HUVECs), it was determined that migration and tube formation of HUVECs increased, as shown in FIG. 17.

### Example <2.3>

### Determination of Vasculogenesis Effect (In Vivo Experiment)

The inventors tested the blood-born hematospheres (BBHSs) generated in Example <2.1> in a preclinical stage and proved a possibility of vasculogenesis.

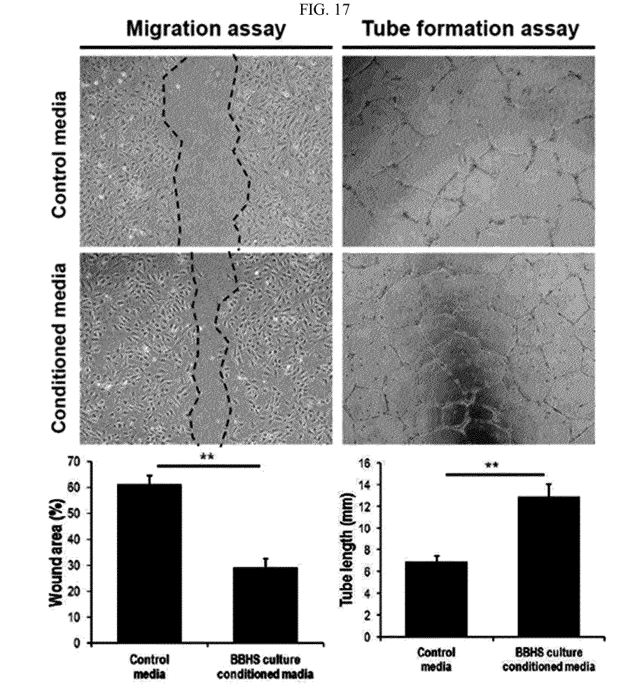

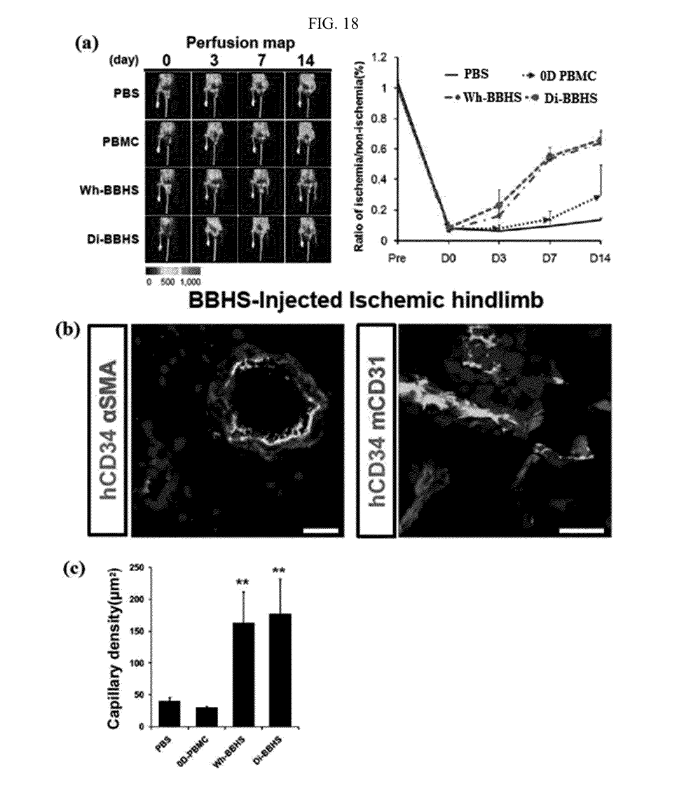

Specifically, ischemia was induced in a hindlimb of a nude mouse having a degraded immune system and blood-born hematospheres of the present invention were injected. Then, before ischemia, immediately after ischemia, on the 3^{rd}, 7^{th}, and 14^{th} day after ischemia, perfusion was measured by laser Doppler perfusion imaging (LDPI). As a result, as shown in FIG. 18A, compared to a group to which a phosphate buffered saline (PBS) or human blood-derived monocytes are directly injected, better perfusion was shown when the blood-born hematospheres generated in Example <2.1> were directly injected or hematospheres were dissociated and injected.

Also, an immunofluorescent technique was performed using antibodies specific to human cells of cluster of differentiation 34 (CD34, green) serving as a vascular endothelial cell marker and alpha smooth muscle actin (SMA-a, red). As a result, as shown in FIG. 18B, it was determined that blood-born hematospheres actually enable vasculogenesis.

In addition, in order to observe blood vessels, BS-1 Lectin staining was performed. As a result, as shown in FIG. 18C, when hematospheres were injected, more blood vessel generation was determined.

The result suggests that vascular endothelial cells and vascular smooth muscle cells may be induced by effective 3D culturing of blood monocyte cells, and thereby vasculogenesis may be efficiently performed. Therefore, when the blood-born hematospheres (BBHSs) generated in Example <2.1> are used, development of a therapeutic agent for ischemic diseases may be expected.

### Example 3

### Example <3.1>

### Generation of Blood-Born Hematospheres (BBHSs)

(1) Step of Isolating Monocyte Cells from Peripheral Blood

Peripheral blood was obtained using a heparin (about 100 ul)-coated 50 ml syringe. 10 ml of the peripheral blood was input to a 50 ml tube. 30 ml of a phosphate-buffered saline (PBS) was added thereto and carefully mixed. 10 ml of Ficoll applied to the diluted blood to express a density gradient was slowly added into the bottom of the tube using a pipet aid. A transparent Ficoll layer and a red blood layer were separated and then centrifuged at 2,500 rpm and 25° C. for 30 minutes with a minimum stop rate. A yellow serum layer, a white monocyte layer, and a transparent Ficoll layer were isolated in an upper portion and a red layer of red blood cells and a polynuclear layer were isolated in a bottom portion. After isolation was confirmed, the yellow serum layer at an upper portion was taken out and then the white monocyte layer was carefully transferred to a new tube. The transferred monocyte layer was divided into two tubes, and the tubes were then filled with PBS and centrifuged at 1,800 rpm and 4° C. for 10 minutes. Cell pellets were suspended into single cells by vortexing and then the tubes were then filled with PBS and centrifuged at 1,700 rpm and 4° C. for 10 minutes. The above washing process was repeated three times to remove substances used to express the density gradient and residual substances in blood. The number of cells was measured using a hemocytometer before the final centrifugation is performed.

### (2) Step of 3D Culturing

Cells that had finished the washing process were suspended in an ultra-low attachment culture dish at a high density of 10⁶/ml or more using a culture solution in which 5% FBS was added to an animal origin material removal culture solution (Stemspan, mTeSR) or endothelial basal medium-2 (EBM-2), were cultured in an incubator to which 5% CO₂ was supplied at 37° C., and the same medium was added to the culture after the first 2 days.

FIG. 19 is a diagram schematically illustrating a process in which peripheral blood mononuclear cells (PBMCs) are isolated and then blood-born hematospheres (BBHSs) are cultured for 5 days.

(3) Step of Dissociating Hematospheres into Single Cells

The hematospheres obtained through the culturing process underwent the process of being isolated to single cells for use in characteristic analysis and treatment. The suspended hematospheres were gathered at a center by horizontally and vertically shaking the culture dish. Only the hematospheres were transferred to a tube under a microscope and centrifuged at 1,700 rpm and 4° C. for 10 minutes. Cell pellets were gently detached using 1 ml of Accutase serving as a cell dissociation solution, and cultured in an incubator at 37° C. for 2 to 3 minutes. Cells of which incubation had finished were added with 1 ml of a cell culture solution and pipetted several times. The tubes were filled with PBS and centrifuged at 1,700 rpm and 4° C. for 10 minutes.

### Example <3.2>

### Analysis of Protein and Gene Characteristics of Lymphatic Vessel-Related Marker in Blood-Born Hematospheres

### (1) Flow Cytometry

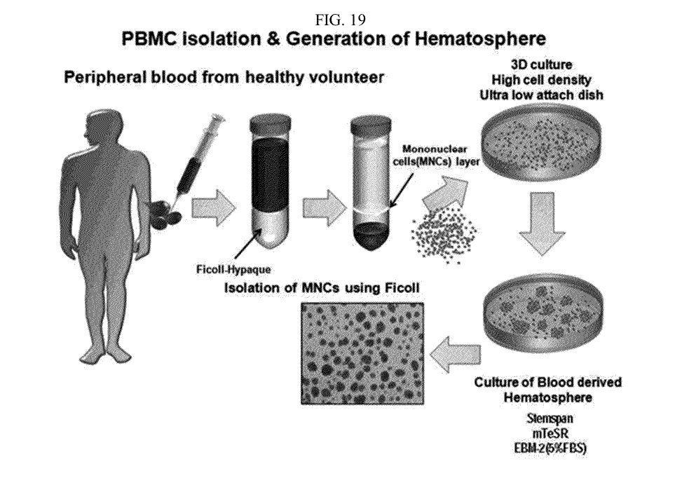

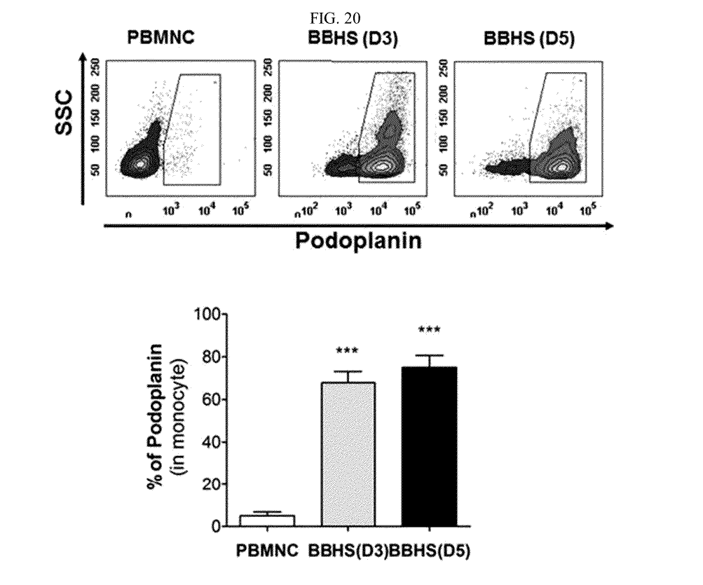

In order to determine expression of podoplanin, which is a representative lymphatic vessel marker in the blood-born hematospheres generated in Example <3.1>, flow cytometry was performed. Monocyte cells isolated from peripheral blood were suspended using PBS including 0.2% bovine serum albumin (BSA) and 1% fetal bovine serum (FBS), and stained with a monocyte marker (CD14) and podoplanin antibodies, and then flow cytometry was performed. Blood-born hematospheres cultured for 3 days and 5 days were dissociated into single cells as described in Example <3.1>, and the same flow cytometry was performed. FIG. 20 shows the results.

As shown in FIG. 20, when 3D culturing was performed, it was determined that podoplanin positive cells in monocytes significantly increased. The result indicates that the 3D culturing is very effective in proliferation of lymphatic vessel marker positive cells.

### (2) Protein Expression Analysis

Expression of representative lymphatic vessel-specific proteins, podoplanin and VEGFR3, in the blood-born hematospheres generated in Example <3.1>, was determined by Western blot and an immunofluorescent technique.

### 1) Western Blot

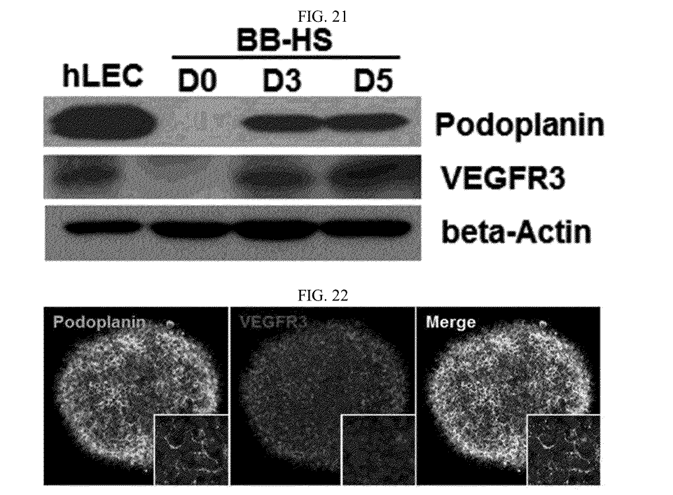

Proteins were extracted from monocyte cells isolated from peripheral blood and 3D cultured blood-born hematospheres, separated by a molecular weight difference using SDS-PAGE, and transferred to a polyvinylidine fluoride (PVDF) membrane. Then, a primary antibody capable of labeling a desired protein and a secondary antibody conjugated with horseradish peroxidase (HRP) with respect to the primary antibody were sequentially attached, and imaged using an X-ray film to analyze expression. FIG. 21 shows the results.

As shown in FIG. 21, when lymphatic vessel-specific proteins, podoplanin and VEGFR3, in monocytes were 3D-cultured, it was determined that expression significantly increased as in human lymphatic endothelial cells (hLECs) serving as a positive control group.

### 2) Immunofluorescence

Blood-born hematospheres cultured for 5 days were attached on a glass slide, fixed using 2% PFA (paraformaldehyde), washed with PBS, and blocked with a 1% BSA solution. Then, a primary antibody capable of labeling a desired protein and a secondary antibody conjugated with fluorescence with respect to the primary antibody were sequentially attached, and expression was analyzed using a confocal microscopy. FIG. 22 shows the results.

As shown in FIG. 22, in blood-born hematospheres, it was determined that lymphatic vessel-specific proteins, podoplanin and VEGFR3, were expressed on a cell surface.

### (3) Gene Expression Analysis

Expression of lymphatic vessel-specific genes in the blood-born hematospheres generated in Example <3.1> was determined

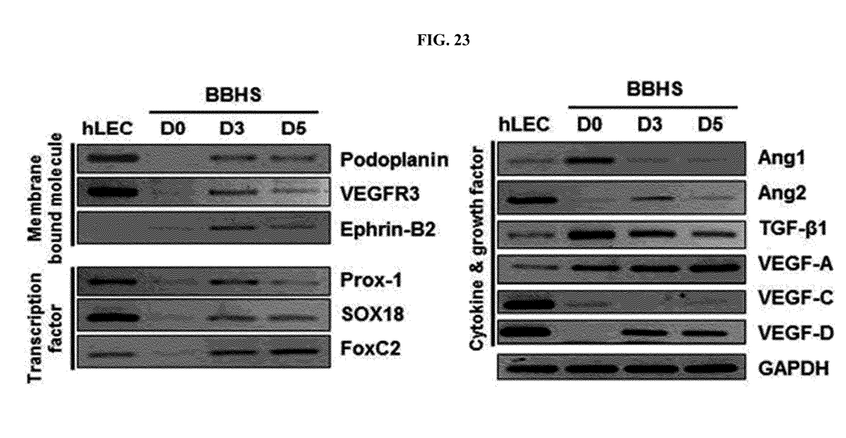

Specifically, monocyte cells isolated from peripheral blood and 3D-cultured blood-born hematospheres were treated with a Trizol reagent to isolate total RNA. cDNA was synthesized using RT-PCR. PCR was performed using podoplanin, VEGFR3, Ephrin-B2, Prox-1, SOX18, FoxC2, Ang1, Ang2, TGF-b1, VEGF-A, VEGF-C, and VEGF-D, and a primer specific to glyceraldehyde 3-phosphate dehydrogenase (GAPDH) genes serving as control genes. The PCR product was analyzed using agarose gel electrophoresis, and expression of these genes was determined FIG. 23 shows the results.

As shown in FIG. 23, in monocyte cells isolated from peripheral blood, expression of lymphatic vessel-specific genes, podoplanin, VEGFR3, Ephrin-B2, Prox-1, SOX18, FoxC2, Ang2, VEGF-A, and VEGF-D, was slight. However, in the blood-born hematospheres (D3 and D5) generated in Example <3.1>, these specific genes were expressed.

Also, it was determined that expression of Ang1 and TGF-b1 decreased in blood-born hematospheres and VEGF-C was slightly expressed before and after the 3D culturing. As a positive control group, human lymphatic endothelial cells (hLECs) were used.

### Example <3.3>

### Isolating Cells to Lymphatic Vessel Marker Podoplanin and Analysis of Characteristics of Isolated Cells

### (1) Isolation of Podoplanin Positive Cells and Negative Cells Using a Cell Sorter by Flow Cytometry

In order to know a difference between positive cells and negative cells of podoplanin, which is a representative lymphatic vessel marker in the blood-born hematospheres generated in Example <3.1>, cells were isolated using a cell sorter using flow cytometry.

Specifically, blood-born hematospheres cultured for 5 days were dissociated into single cells as described in Example <3.1>, and then stained with a lymphatic vessel marker podoplanin. Positive cells and negative cells were isolated using a cell sorter by flow cytometry.

### (2) Gene Expression Analysis

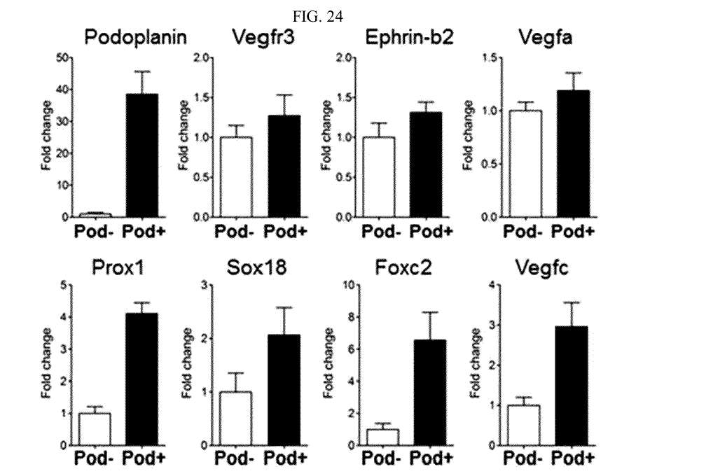

In order to compare gene expression, the cells isolated using a cell sorter by flow cytometry were treated with a Trizol reagent to isolate total RNA. cDNA was synthesized using RT-PCR. Then, real time-PCR was performed using podoplanin, VEGFR3, Ephrin-B2, Prox-1, SOX18, FoxC2, VEGF-A, and VEGF-C, and a primer specific to GAPDH genes serving as control genes. FIG. 24 shows the results.

As shown in FIG. 24, it was determined that expression of lymphatic vessel-specific genes, podoplanin, Prox-1, SOX18, FoxC2, and VEGF-C, significantly increased in podoplanin positive cells compared to podoplanin negative cells. On the other hand, it was determined that expression of VEGFR3, Ephrin-B2, and VEGF-A showed a slight difference between positive cells and negative cells.

### Example <3.4>

### Analysis of Neolymphangiogenesis Effect of Pharmaceutical Composition in Body

### (1) Analysis of Neolymphangiogenesis Effect Through Degree of Wound Healing in Wound Healing Model

In order to analyze a lymphangiogenesis effect in a body of the blood-born hematospheres generated in Example <3.1>, a wound healing model of immunodeficient mice was used. A back and an ear of the immunodeficient mice were punched. Then, cells dissociated into single cells as described in Example <3.1>, and isolated platelets were injected into near the wound. Groups injected into the wound included a PBS group in which no cells were injected, a group in which only platelets were injected, a group in which only blood-born hematospheres were injected, a group in which blood-born hematospheres and platelets were simultaneously injected, and a group in which podoplanin neutralizing antibody-added blood-born hematospheres and platelets were simultaneously injected.

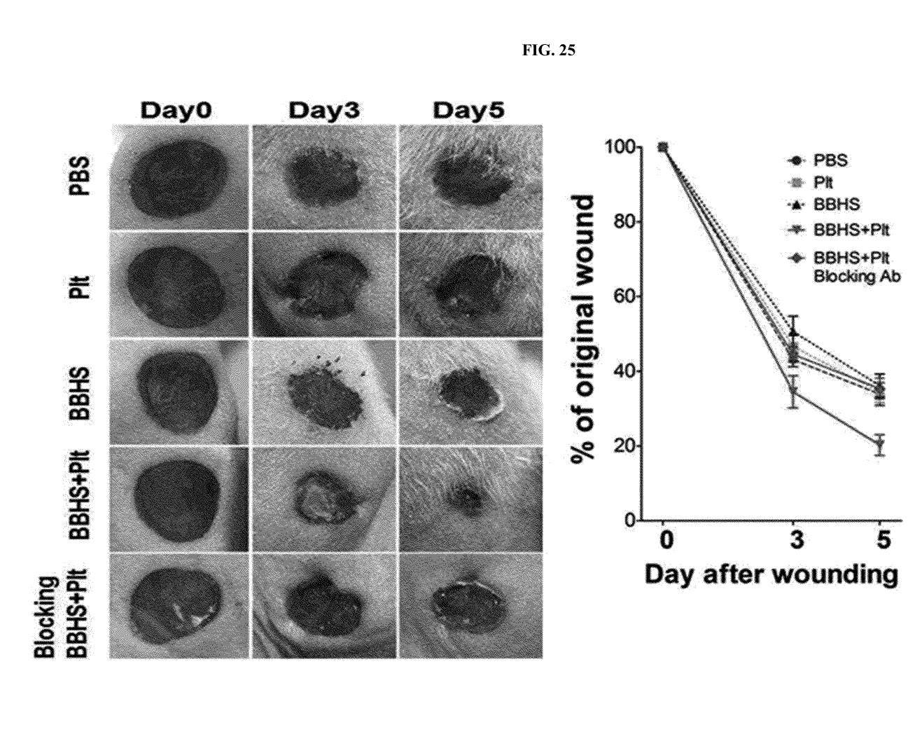

As shown in FIG. 25, in pictures of imaging a degree of back wound healing of the immunodeficient mice by time and a quantitative graph, it was determined that a wound healing effect significantly increased in the group in which blood-born hematospheres and platelets were simultaneously injected.

### (2) Analysis of Neolymphangiogenesis Effect Through Section Staining of Wound Healing Model Tissue

In order to prove that a wound healing effect of the group in which the blood-born hematospheres generated in Example <3.1> and platelets were simultaneously injected is facilitated by lymphangiogenesis, sections of tissues of back and ear wounds were immunofluorescent-stained with a lymphatic vessel marker, a lymphatic vessel endothelial receptor 1 (LYVE-1), and the results were compared and analyzed. Back tissue was fixed in an optimal cutting temperature (OCT) compound, and one section was attached to a glass slide. Front and rear sections of ear tissue were separated under a dissecting microscope, and an inside was fixed to stain. Then, expression was analyzed using a confocal microscopy. FIGS. 26 and 27 show the results.

As shown in FIG. 26, it was determined that lymphatic vessels significantly increased when the group in which the blood-born hematospheres cultured according to the present invention and platelets were simultaneously injected was put into a back wound of the immune-deficient mice, compared to the group in which only blood-born hematospheres were injected and the group in which podoplanin neutralizing antibody-added blood-born hematospheres and platelets were simultaneously injected.

Also, as shown in FIG. 27, it was determined that bifurcation of the lymphatic vessels significantly increased when the group in which blood-born hematospheres cultured according to the present invention and platelets were simultaneously injected was put into an ear wound of the immune-deficient mice, compared to the group in which only blood-born hematospheres were injected and the group in which podoplanin neutralizing antibody-added blood-born hematospheres and platelets were simultaneously injected.

### Example 4

### Example <4.1>

### Generation of Blood-Born Hematospheres (BBHSs)

(1) Step of Isolating Monocyte Cells from Peripheral Blood

Peripheral blood was obtained using a heparin (about 100 ul)-coated 50 ml syringe. 10 ml of the peripheral blood was input to a 50 ml tube. 30 ml of a phosphate-buffered saline (PBS) was added thereto and carefully mixed. 10 ml of Ficoll applied to the diluted blood to express a density gradient was slowly added into the bottom of the tube using a pipet aid. A transparent Ficoll layer and a red blood layer were separated and then centrifuged at 2,500 rpm and 25° C. for 30 minutes with a minimum stop rate. A yellow serum layer, a white monocyte layer, and a transparent Ficoll layer were isolated in an upper portion and a red layer of red blood cells and a monocyte layer were isolated in a bottom portion. After isolation was confirmed, the yellow serum layer at an upper portion was taken out and then the white monocyte layer was carefully transferred to a new tube. The transferred monocyte layer was divided into two tubes, and the tubes were then filled with PBS and centrifuged at 1,800 rpm and 4° C. for 10 minutes. Cell pellets were suspended into single cells by vortexing and then the tubes were then filled with PBS and centrifuged at 1,700 rpm and 4° C. for 10 minutes. The above washing process was repeated three times to remove substances used to express the density gradient and residual substances in blood. The number of cells was measured using a hemocytometer before the final centrifugation is performed.

### (2) Step of 3D Culturing

Cells that had finished the washing process were suspended in an ultra-low attachment culture dish at a high density of 10⁶/ml or more using a culture solution in which 5% FBS was added to an endothelial basal medium-2 (EBM-2), and cultured in an incubator to which 5% CO₂ was supplied at 37° C., and the same medium was added to the culture after the first 2 days.

FIG. 1 is a diagram schematically illustrating a process in which peripheral blood mononuclear cells (PBMCs) are isolated and then blood-born hematospheres (BBHSs) are cultured for 5 days.

(3) Step of Dissociating Hematospheres into Single Cells

The hematospheres obtained through the culturing process underwent the process of being isolated to single cells for use in characteristic analysis and treatment. The suspended hematospheres were gathered at a center by horizontally and vertically shaking the culture dish. Only the hematospheres were transferred to a tube under a microscope and centrifuged at 1,700 rpm and 4° C. for 10 minutes. Cell pellets were gently detached using 1 ml of Accutase serving as a cell dissociation solution, and cultured in an incubator at 37° C. for 2 to 3 minutes. Cells of which incubation had finished were added with 1 ml of a cell culture solution and pipetted several times. The tubes were filled with PBS and centrifuged at 1,700 rpm and 4° C. for 10 minutes.

### Example <4.2>

### Determination of Nerve Cell Differentiation in Blood-Born Hematospheres

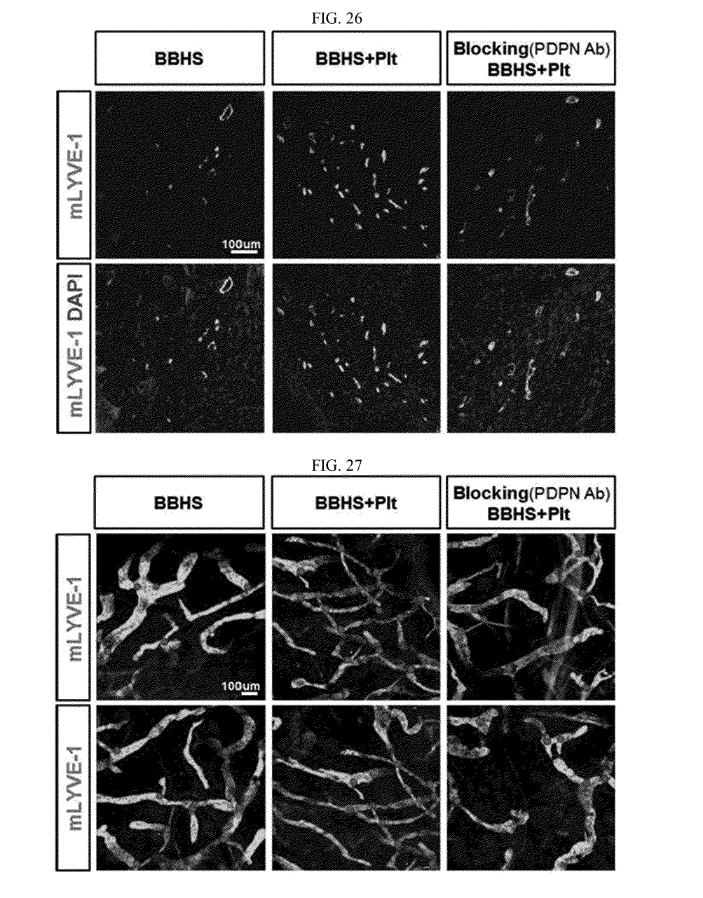

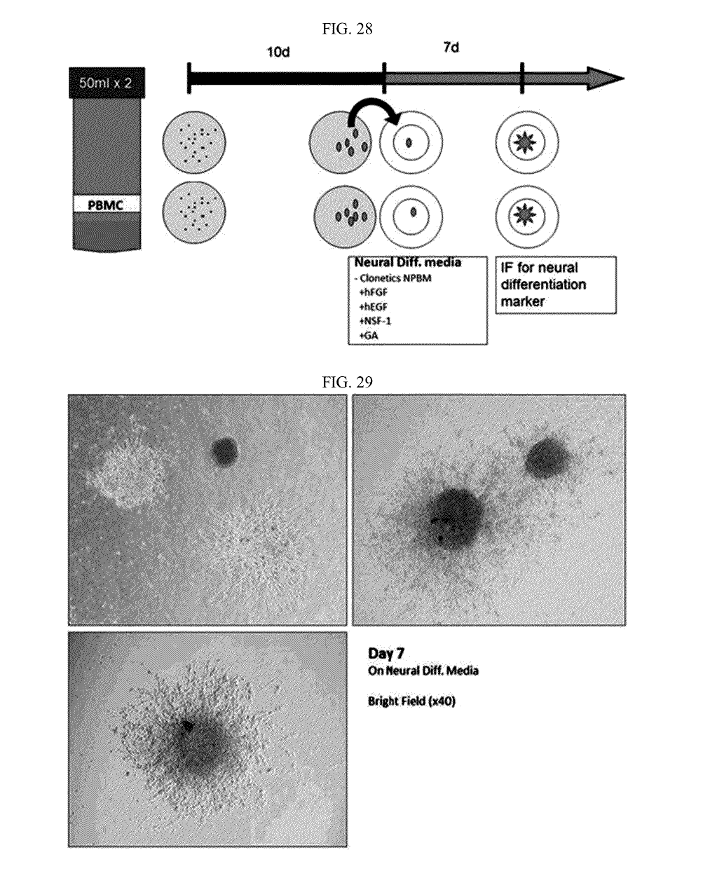

The hematospheres (BBHSs) generated in Example <4.1> were induced to differentiate into nerve cells as shown in a schematic diagram of FIG. 28.

Specifically, human peripheral blood monocytes were 3D-cultured for 10 days to generate blood-born hematospheres. Then, the hematospheres were transferred to a general culture dish other than the ultra-low attachment culture dish and then a nerve cell differentiation medium (Clonetics NPBM, Lonza) was added with hFGF, hEGF, NSF-1, and GA. The hematospheres were cultured for 7 days and imaged using an Olympus IX2 inverted fluorescence microscope (Olympus, Tokyo, Japan) device in which an Olympus DP50 CF CCD camera is installed. FIG. 29 shows the captured images. As a result, it can be seen that most blood-born hematospheres well differentiated into nerve cells.

### Example <4.3>

### Protein Characteristic Analysis after Nerve Cell Differentiation Induction

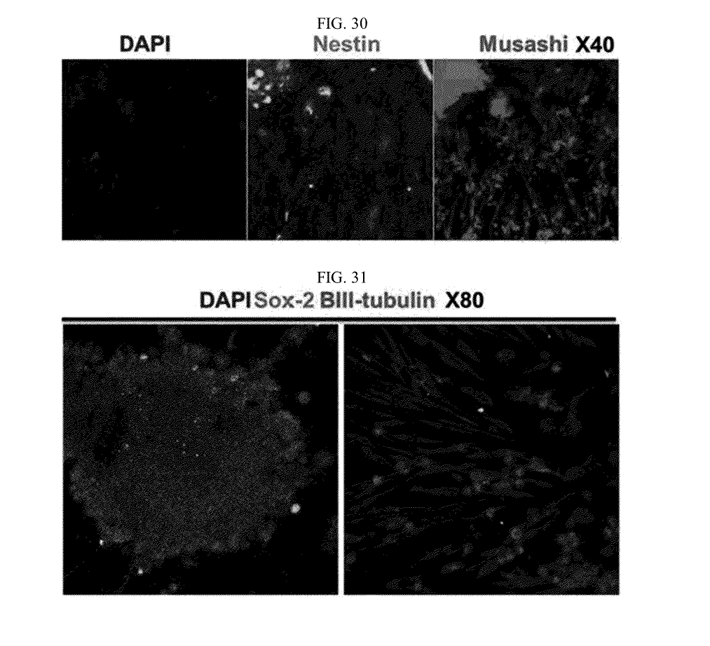

After the blood-born hematospheres were generated, differentiation of nerve cells was induced using the method in Example <4.2>. An immunofluorescence assay was used to determine whether neural progenitor cells were actually induced. FIG. 30 shows the results.

As shown in FIG. 30, Nestin (green) known as a marker of neural progenitor cells was partially stained, and Musashi (red) which is an another marker of neural progenitor cells was mostly stained. DAPI (blue) was used for nuclear staining.

In addition, an immunofluorescence assay was used to determine whether blood-born hematospheres are generated and then differentiate into nerve cells through nerve cell differentiation induction. FIG. 31 shows the results.

As shown in FIG. 31, Sox2 (green) maintaining an undifferentiated state of neural stem cells was partially expressed. However, it can be seen that beta-III tubulin (red), which is a representative marker of nerve cells, was expressed in most nerve cells derived from blood-born hematospheres.

The result indicates that neural progenitor cell induction and nerve cell differentiation are possible in blood-born hematospheres. Eventually, the nerve cells are expected to be used for treatment of neurological diseases and further expected to be used for development of a novel cell therapeutic agent for treating neurological diseases.

### Example 5

According to the present invention, blood-born hematospheres (BBHSs) were generated (Example <5.1>), expression of insulin in the generated blood-born hematospheres (BBHSs) was determined (Example <5.2>), differentiation of insulin secreting cell was induced (Example <5.3>), and finally, genes actually important for insulin or insulin expression were determined and actual secretion of insulin was determined (Example <5.4>).

### Example <5.1>

### Generation and Characteristics of Blood-Born Hematospheres (BBHS)

(1) Step of Isolating Monocyte Cells from Peripheral Blood

Peripheral blood was obtained using a heparin (about 100 ul)-coated 50 ml syringe. 10 ml of the peripheral blood was input to a 50 ml tube. 30 ml of a phosphate-buffered saline (PBS) was added thereto and carefully mixed. 10 ml of Ficoll applied to the diluted blood to express a density gradient was slowly added into the bottom of the tube using a pipet aid. A transparent Ficoll layer and a red blood layer were separated and then centrifuged at 2,500 rpm and 25° C. for 30 minutes with a minimum stop rate. A yellow serum layer, a white monocyte layer, and a transparent Ficoll layer were isolated in an upper portion and a red layer of red blood cells and a monocyte layer were isolated in a bottom portion. After isolation was confirmed, the yellow serum layer at an upper portion was taken out and then the white monocyte layer was carefully transferred to a new tube. The transferred monocyte layer was divided into two tubes, and the tubes were then filled with PBS and centrifuged at 1,800 rpm and 4° C. for 10 minutes. Cell pellets were suspended into single cells by vortexing and then the tubes were then filled with PBS and centrifuged at 1,700 rpm and 4° C. for 10 minutes. The above washing process was repeated three times to remove substances used to express the density gradient and residual substances in blood. The number of cells was measured using a hemocytometer before the final centrifugation is performed.

### (2) Step of 3D Culturing

Cells that had finished the washing process were suspended in an ultra-low attachment culture dish at a high density of 10⁶/ml or more using a culture solution in which 5% FBS was added to an endothelial basal medium-2 (EBM-2), and cultured in an incubator to which 5% CO₂ was supplied at 37° C., and the same medium was added to the culture after the first 2 days.

FIG. 1 is a diagram schematically illustrating a process in which peripheral blood mononuclear cells (PBMCs) are isolated and then blood-born hematospheres (BBHSs) are cultured for 5 days.

(3) Step of Dissociating Hematospheres into Single Cells

The hematospheres obtained through the culturing process underwent the process of being isolated to single cells for use in characteristic analysis and treatment. The suspended hematospheres were gathered at a center by horizontally and vertically shaking the culture dish. Only the hematospheres were transferred to a tube under a microscope and centrifuged at 1,700 rpm and 4° C. for 10 minutes. Cell pellets were gently detached using 1 ml of Accutase serving as a cell dissociation solution, and cultured in an incubator at 37° C. for 2 to 3 minutes. Cells of which incubation had finished were added with 1 ml of a cell culture solution and pipetted several times. The tubes were filled with PBS and centrifuged at 1,700 rpm and 4° C. for 10 minutes.

### Example <5.2>

### Determination of Insulin Secreting Cells in Blood-Born Hematospheres

In order to determine a level of insulin expression of the blood-born hematospheres generated according to the present invention in FIG. 32, insulin was stained using immunofluorescence. As a result, some cells (Nestin and beta-tubulin) expressing insulin were determined.

Specifically, the immunofluorescence was performed such that blood-born hematospheres were fixed for 30 minutes, washed with PBS three times, blocked for 30 minutes, and stained using an insulin antibody (green: insulin and blue: nucleus).

It is known that neural progenitor cells and nerve cells expressing Nestin and beta-tubulin share many parts with insulin progenitor cells and easily differentiate into insulin secreting cells (IPCs) (Hori Y, Gu X, Xie X, Kim SK. Differentiation of insulin-producing cells from human neural progenitor cells. PLoS Med. 2005).

Therefore, the immunofluorescence assay as in FIG. 32 was performed using Nestin and beta-tubulin antibodies. Expression levels of Nestin and beta-tubulin in blood-born hematospheres were analyzed using a confocal microscope.

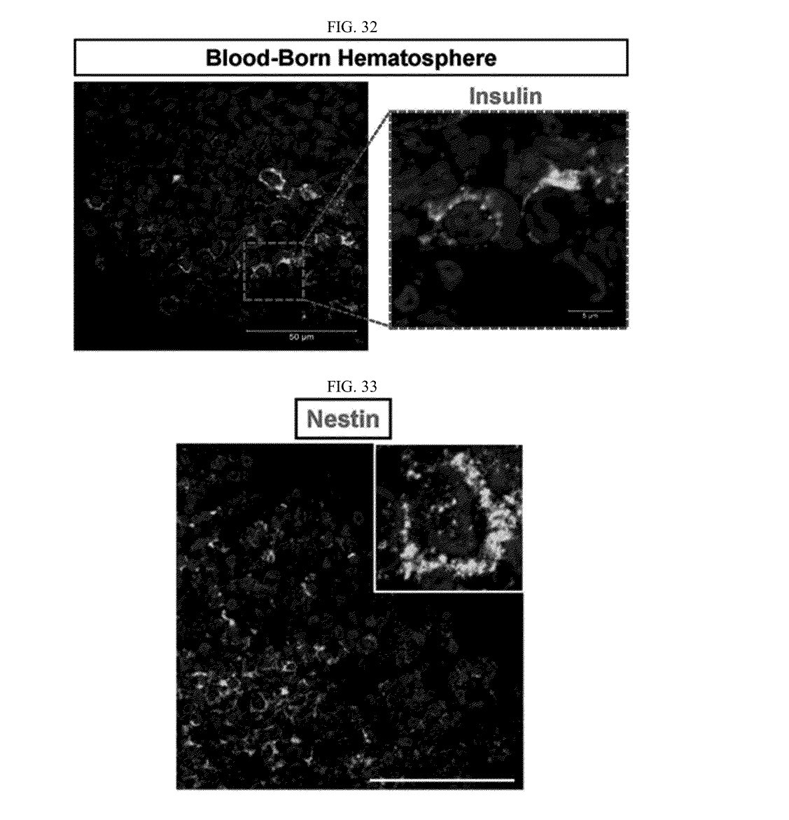

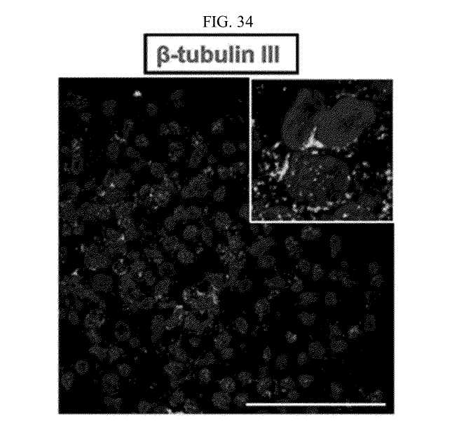

As a result, it was proved that there are many cells expressing Nestin (refer to FIG. 33) and beta-tubulin (refer to FIG. 34) in blood-born hematospheres (FIG. 33—green: Nestin, FIG. 34—green: beta-tubulin, blue: nucleus, and Scale bar: 50 um).

### Example <5.3>

### Insulin Secreting Cell Differentiation Induction in Blood-Born Hematospheres

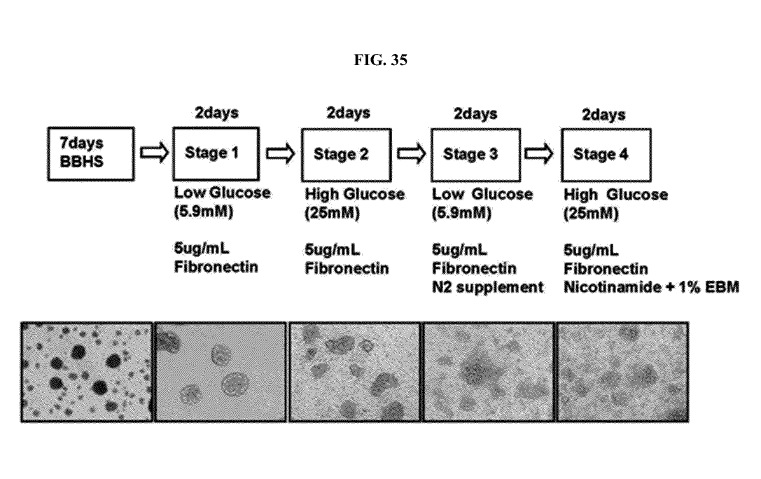

FIG. 35 illustrates a process in which blood-born hematospheres generated according to the present invention were cultured for 7 days and then differentiated into insulin secreting cells.

More specifically, first, in a first step, blood-born hematospheres were cultured for 7 days, the blood-born hematospheres (BBHSs) were transferred to a Fibronectin (5 ug/ml)-coated culture dish, and cultured for 2 days using a medium in which 1% FBS and Low-Glucose (5.9 mM) were added to EBM-2. In a second step, the medium was changed to a medium in which 1% FBS and high-glucose (25 mM) were added to EBM-2 and cultured for 2 days. In a third step, the medium was changed to a medium in which 1% FBS, Low-Glucose (5.9 mM), and an N2 supplement (Invitrogen) were added to EBM-2 and then cultured for 2 days. Finally, in a fourth step, the medium was changed to a medium in which 1% FBS, High-Glucose (25 mM), and 10 mM of Nicotinamide (Sigma Aldrich) were added to EBM-2 and cultured for 2 days.

### Example <5.4>

### Analysis of Genes and Protein Characteristics after Insulin Secreting Cell Differentiation Induction

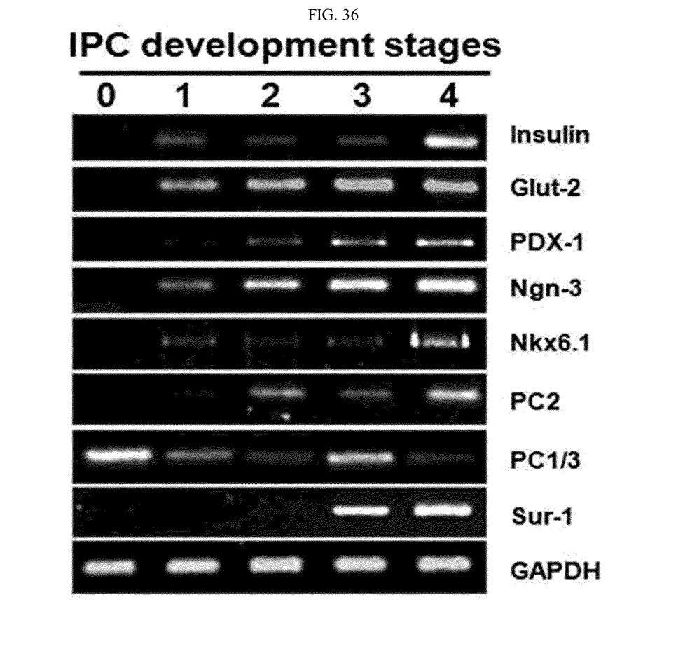

In each step of differentiating blood-born hematospheres (BBHSs) generated according to the present invention into insulin secreting cells, RT-PCR was used to synthesize cDNA. Then, PCR was performed using primers specific to glucose transporter 2 (GLUT2), insulin promoter factor 1 (Pdx-1), Neurogenin 3 (Ngn3), Nkx6.1, Proprotein convertase 2 (PC2), PC1/3, SUR1, and GAPDH genes which are genes important for insulin and insulin secretion. The PCR product was analyzed using agarose gel electrophoresis, and expression of these genes was determined FIG. 36 shows the results.

Specifically, the most important insulin genes increased. Insulin promoter factor 1 (Pdx-1), Neurogenin 3 (Ngn3), and Nkx6.1, which are known as transcription factor genes important for development of beta cells of pancreas, increased in each step. Also, glucose transporter 2 (GLUT2) and proprotein convertase 2 (PC2), which are important for an endocrine function of pancreas, increased in each step. Also, Kir6.2 and ATP-binding cassette transporter sub-family C member 8 (SUR1), which are important for beta cells, also increased in each step. Primers used in this case are shown in Table. 2.

TABLE 2

| | | SEQ | | Product |
|---|---|---|---|---|
| | | ID | Access | size |
| Primer | Sequence | NO | Number | (bp) |
| Insulin | Forward 5′-CCTGTGCGGCTCACACCTGG-3′ | 17 | NM_000207 | 540 |
| | Reverse 5′-CCACTCAGGCAGGCAGCCAC-3′ | 18 | | |
| Glut2 | Forward 5′-AGGACTTCTGTGGACCTTATGTG-3′ | 19 | L09674 | 231 |
| Glut2 | Reverse 5′-GTTCATGTCAAAAAGCAGGG-3′ | 20 | L09674 | 231 |
| Pdx1 | Forward | 21 | NM_000209 | 220 |
| | 5′-GGATGAAGTCTACCAAAGCTCACGC-3′ | | | |
| Pdx1 | Reverse 5′-CCAGATCTTGATGTGTCTCTCGGT | 22 | NM_000209 | 220 |
| | C-3′ | | | |
| Ngn3 | Forward 5′-CGTGAACTCCTTGAACTGAGCAG-3′ | 23 | AF234829 | 211 |
| Ngn3 | Reverse 5′-TGGCACTCCTGGGACAGATTTC-3′ | 24 | AF234829 | 211 |
| Nkx6.1 | Forward 5′-CAATGGAGGGCACCCGGCAG-3′ | 25 | NM_006168.2 | 599 |
| Nkx6.1 | Reverse 5′-CCAGAAGATGGGCGTCCGGC-3′ | 26 | NM_006168.2 | 599 |
| PC2 | Forward 5′-GCATCAAGCACAGACCTACACTCG-3′ | 27 | NM_002594 | 309 |
| PC2 | Reverse 5′-GAGACACAACCCTTCATCCTTC-3′ | 28 | NM_002594 | 309 |
| PC1/3 | Forward | 29 | NM_000439 | 457 |
| | 5′-TTGGCTGAAAGAGAACGGGATACATCT-3′ | | | |
| | Reverse | 30 | | |
| | 5′-ACTTCTTTGGTGATTGCTTTGGCGGTG-3′ | | | |
| SUR1 | Forward 5′-CACATCCACCACAGCACATGG-3′ | 31 | NM_000352.3 | 420 |
| SUR1 | Reverse | 32 | NM_000352.3 | 420 |
| | 5′-GTCTTGAAGAAGATGTATCTCCTCA-3′ | | | |
| GAPDH | Forward 5′-CAAATTCGTTGTCATACCAG-3′ | 33 | NM_002046 | 480 |
| GAPDH | Reverse 5′-CGTGGAAGGACTCATGAC-3′ | 34 | NM_002046 | 480 |

In FIG. 37, blood-born hematospheres (BBHSs) were induced to differentiate into insulin secreting cells, and expression of insulin and Nestin was determined using an immunofluorescence assay. The immunofluorescence result showed that insulin and Nestin were expressed. Also, compared to blood-born hematospheres, when blood-born hematospheres were induced to differentiate into insulin secreting cells, cells expressing insulin more increased (red: insulin, green: Nestin, blue: nucleus, and Scale bar: 50 um).

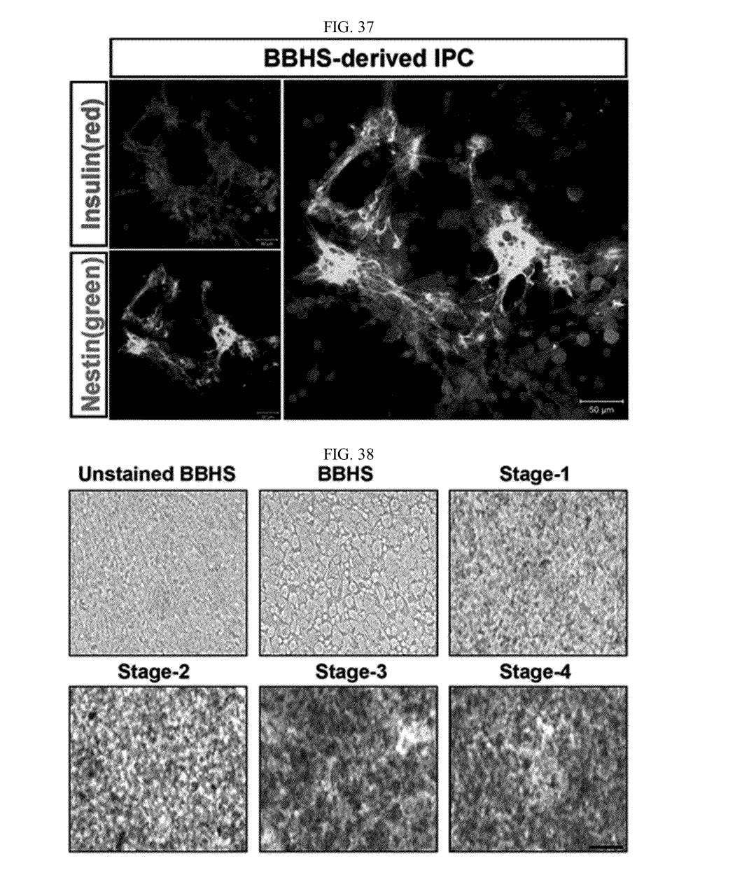

In FIG. 38, by dithizone staining (DTZ) in which zinc ions were detected in insulin molecules in insulin secreting cells and the cells are stained with red (Crimson red), actual expression of a large amount of insulin was determined after insulin secreting cell differentiation (Scale bar: 10 um). DTZ (100 ug/ml) was added to an EBM-2 culture solution in which 1% FBS was included and DTZ staining was used in each step of insulin secreting cell differentiation.

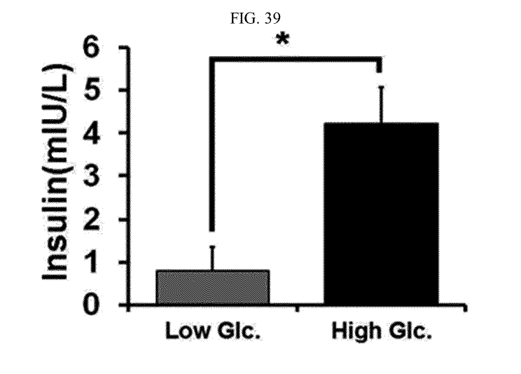

In FIG. 39, in order to determine whether insulin is actually secreted, glucose-stimulated insulin secretion (GSIS) was analyzed using supernatants of cells through ELISA.

More specifically, the analysis of GSIS was performed such that blood-born hematospheres (BBHSs) were induced to differentiate into insulin secreting cells, washed with PBS once and incubated for 1 hour at 37° C. using a Krebs-Ringer bicarbonate (KRB) buffer (120 mM NaCl, 5 mM KCl, 2.5 mM CaCl₂, 1.1 mM MgCl₂, 25 mM NaHCO₃, and 0.1 g BSA) in which low glucose (5.9 mM) was included. Then, the supernatants were removed, and a new KRB buffer including 5.9 mM (low glucose) or 25 mM of glucose (high glucose) was added into the culture and then incubated for 2 hours at 37° C. The supernatants were obtained and a degree of insulin secretion was analyzed through ELISA. As a result, it was determined that more insulin was secreted in high glucose than low glucose. That is, it was determined that insulin secreting cells derived from blood-born hematospheres (BBHSs) secrete insulin by glucose stimulation (refer to FIG. 39).

The above description of the invention is only exemplary, and it will be understood by those skilled in the art that various modifications can be made without departing from the scope of the present invention and without changing essential features. Therefore, the above-described examples should be considered in a descriptive sense only and not for purposes of limitation.

A pharmaceutical composition for treating immune-related diseases, a pharmaceutical composition for treating ischemic diseases, a pharmaceutical composition for promoting lymphangiogenesis, a pharmaceutical composition for treating neurological diseases, a pharmaceutical composition for treating metabolic diseases, and the like, which contain blood-born hematospheres according to the present invention, may differentiate into anti-inflammatory monocyte cells, vascular endothelial cells and vascular smooth muscle cells, lymphatic vessel adult stem cells and progenitor cells, neural progenitor cells and nerve cells, insulin secreting cells, and the like by effective 3D culturing using blood monocyte cells, and thereby may be used for development of a cell therapeutic agent for various types of diseases.

1. A pharmaceutical composition for promoting lymphatic neovascularization, containing blood-born hematospheres.

2. The pharmaceutical composition of claim 1, wherein the blood-born hematospheres are generated when mononcyte cells are isolated from human blood and then 3D aggregate cultured.

3. The pharmaceutical composition of claim 1,
wherein the blood-born hematospheres are isolated into single cells and then used.

4. The pharmaceutical composition of claim 1,
wherein the blood-born hemastospheres include lymphatic vessel adult stem cells and progenitor cells.

5. The pharmaceutical composition of claim 1, further include platelets.

6. The pharmaceutical composition of claim 1,
wherein the pharmaceutical composition are used for healing wounds.

7. The pharmaceutical composition of claim 1,
wherein the pharmaceutical composition are used for treating diseases including lymphatic dysplasia or other lymphatic disorders.

8. A method of treating wounds by administering the pharmaceutical composition of claim 1 to a subject.

9. A method of treating diseases having lymphatic dysplasia or other lymphatic disorders by administering the pharmaceutical composition of claim 1 to a subject.
